(19) United States
(12) Patent Application Publication
(10) Pub. No.: US 2014/0169533 A1
(43) Pub. Date: Jun. 19, 2014

(54) SYSTEM AND METHOD FOR CAPTURING MEDICAL IMAGES

(71) Applicant: Michael R. Razzano, Marietta, GA (US)

(72) Inventor: Michael R. Razzano, Marietta, GA (US)

(21) Appl. No.: 14/132,048

(22) Filed: Dec. 18, 2013

Related U.S. Application Data
(60) Provisional application No. 61/739,225, filed on Dec. 19, 2012.

(51) Int. Cl. A61B 6/00

(52) U.S. Cl. CPC A61B 6/587
USPC 378/205

(57) ABSTRACT

Disclosed is an alignment device for interfacing a sensor with an X-ray device that includes a body that can be aligned with an X-ray device, a receptor holder that holds the sensor, and an alignment arm that is slidingly connected to the body to permit the position of the receptor holder to be moved to different axial positions with respect to the body.

### CROSS REFERENCE TO RELATED APPLICATIONS

This application claims the benefit of U.S. Provisional Application No. 61/739,225, filed Dec. 19, 2012, under 35 USC 119(e), which is hereby incorporated by reference.

### BACKGROUND

In today's medical profession, there are various ways to capture images of patients, such as images captured for diagnostic purposes. For example, a medical professional such as a dentist can use a traditional x-ray device to capture a film-based x-ray image of the patient's mouth. Medical professionals can also capture an x-ray image in a digital fashion using a digital x-ray device that has a computer workstation and a sensor. Digital cameras are also used by medical professionals to capture still and video images for later storage on a computer in the patient record. Each of the devices and systems typically require separate systems and pieces of equipment. There is a need for improved devices, systems and methods for capturing images.

### BRIEF DESCRIPTION OF THE DRAWINGS

FIG. 1 is a perspective view of an image capture device with an x-ray collimator tube of one embodiment of the present invention.

FIG. 2 is a front end view of a collimator tube of one embodiment of the current invention.

FIG. 3 is perspective view of an intra-oral camera that can be connected to the collimator tube in one embodiment of the current invention.

FIG. 4 is a partial perspective view of a collimator tube of one shape being converted to a collimator tube of another shape using a collimator tube adapter in one embodiment of the current invention.

FIG. 5 is a perspective view of a collimator tube and an insert in one embodiment of the present invention.

FIG. 6 is a front end view of an x-ray device without a collimator tube attached for one embodiment of the present invention.

FIG. 7 is a partial elevational view of a collimator tube mating with the x-ray device of FIG. 6 in a manner that allows rotation of the collimator tube.

FIG. 8 is a side perspective view of a first embodiment receptor holder of the current invention.

FIG. 9 is a back end view of a connection end of the receptor holder of FIG. 10.

FIG. 10 is a partial cross-section view of the connection end of the receptor holder of FIG. 10.

FIG. 11 is a side perspective view of a second embodiment receptor holder of the current invention.

FIG. 12 is a perspective view of an image capture device illustrating an x-ray generator, collimator tube, and receptor holder of one embodiment of the present invention.

FIG. 13 is a side perspective view of an image capture device illustrating an x-ray generator, collimator tube, and receptor holder of one embodiment of the present invention.

FIG. 14 is a diagrammatic view of a system of one embodiment of the present invention for use with the image capture device of FIGS. 1-13.

FIG. 15 illustrates a high-level process flow diagram for the system of FIG. 1 and the image capture device of FIGS. 1-13.

FIG. 16 is a side perspective view of an image capture device illustrating an x-ray generator, collimator tube, and receptor holder of one embodiment of the present invention.

FIG. 17 is a perspective view of an image capture device illustrating an x-ray generator, collimator tube, and receptor holder of one embodiment of the present invention.

FIG. 18 is a perspective view of a connection end of a receptor holder of one embodiment of the present invention.

FIG. 19 is a perspective view of a receptor holder of one embodiment of the present invention.

FIG. 20 is a schematic system diagram of an image capture system of one embodiment of the present invention.

FIG. 21 is a schematic system diagram of an image capture system of one embodiment of the present invention.

FIG. 22 is a schematic system diagram of an image capture system of one embodiment of the present invention.

FIG. 23 is a schematic system diagram of an image capture system of one embodiment of the present invention.

FIG. 24 is a schematic system diagram of an image capture system of one embodiment of the present invention.

FIG. 25 is a schematic system diagram of an image capture system of one embodiment of the present invention.

FIG. 26 is a schematic system diagram of an image capture system of one embodiment of the present invention.

FIG. 27 is a perspective view of one embodiment of a gridded insert.

FIG. 28 is a perspective view of one embodiment of a gridded insert.

FIG. 29 is a perspective view of one embodiment of a gridded insert.

FIG. 30 is a perspective view of one embodiment of a gridded insert.

FIG. 31 is a perspective view of one embodiment of an image receptor holder.

FIG. 32 is a perspective view of one embodiment of an image receptor holder.

FIG. 33 is a perspective view of one embodiment of a mounting pin assembly and image receptor having recesses.

FIG. 34 is a perspective view of one embodiment of a mounting pin assembly.

FIG. 35 is a perspective view of one embodiment of a mounting pin assembly.

FIG. 36 is a perspective view of one embodiment of a mounting pin assembly.

FIG. 37 is an exploded view of one embodiment of a mounting pin assembly.

FIG. 37a is a top view of one embodiment of a mounting pin assembly.

FIG. 38 is a perspective view of one embodiment of an x-ray system having a bite block.

FIG. 39 is a side view of one embodiment of a bite block.

FIG. 39a is a side view of one embodiment of a bite block.

FIG. 40 is a side view of one embodiment of a bite block.

FIG. 40a is a side view of one embodiment of a bite block.

FIG. 40c is a perspective view of one embodiment of a bite block and mounting pin assembly.

FIG. 40d is a top view of one embodiment of a bite block.

FIG. 40e is a perspective view of one embodiment of a bite block.

FIG. 40f is a perspective view of one embodiment of a bite block mounted to a mounting pin assembly.

FIG. 40g is a perspective view of one embodiment of a finger grip.

FIG. 41 is a perspective view of one embodiment of molded blocks useful in adapting image receptors to pin-type connections.

FIG. 42 is a perspective view of one embodiment of vinyl pockets useful in adapting image receptors to pin-type connections.

FIG. 43 is a perspective view of one embodiment of a carrier card useful in orienting vinyl pockets prior to fixation to an image receptor.

FIG. 44 is a perspective view of one embodiment of vinyl pockets after adhesion to an image receptor.

FIG. 45 is a perspective view of one embodiment of a pocket assembly.

FIG. 46 is a perspective view of one embodiment of a pocket assembly and image receptor.

FIG. 46a is a perspective view of one embodiment of a lattice assembly.

FIG. 47 is a perspective view of one embodiment of a pocket assembly having a cover.

FIG. 48 is a perspective view of one embodiment of a pocket assembly packaged in a sterile pack.

FIG. 49 is a perspective view of one embodiment of wire holder clips.

FIG. 50 is a perspective view of one embodiment of a shaped alignment bar.

FIG. 50a is a front view of one embodiment of a collimator tube having divot tracks.

FIG. 50b is a perspective view of one embodiment of an alignment ring having divots.

FIG. 51 is a perspective view of one embodiment of an image receptor adapted to receive another image receptor.

FIG. 52 is a side view of an image receptor adapted to receive another image receptor.

FIG. 53 is a front view of a collimator tube.

FIG. 54 is a perspective view of one embodiment of an alignment ring.

FIG. 55 is a perspective view of one embodiment of an alignment ring.

FIG. 56 is a perspective view of one embodiment of an alignment ring.

FIG. 57 is a top view of one embodiment of an alignment ring.

FIG. 58 is a perspective view of one embodiment of an alignment ring.

FIG. 59 is a perspective view of one embodiment of an alignment ring.

FIG. 60 is a front view of one embodiment of an alignment ring.

FIG. 61 is a perspective view of one embodiment of bar of an x-ray device.

FIG. 62 is a perspective view of one embodiment of bitewing.

FIG. 63 is a top view of one embodiment of bitewing.

FIG. 64a is a perspective view of one embodiment of bitewing.

FIG. 64b is a perspective view of one embodiment of a replaceable mounting pin.

FIG. 65 is a perspective view of one embodiment of bitewing.

### DETAILED DESCRIPTION

Reference will now be made to certain embodiments and specific language will be used to describe the same. It will nevertheless be understood that no limitation of the scope of this disclosure and the claims are thereby intended, such alterations, further modifications and further applications of the principles described herein being contemplated as would normally occur to one skilled in the art to which this disclosure relates. In several figures, where there are the same or similar elements, those elements are designated with the same or similar reference numerals.

The present disclosure relates to systems and methods for capturing medical images. A collimator tube of the system includes one or more integrated devices, such as a radio frequency transmitter, a camera, a frame grabber, or a transilluminator light. In one embodiment, the integrated devices are integrated within one or more walls of the collimator tube. The integrated devices are used instead of or in addition to an x-ray generator, which is coupled to the collimator tube. The camera is used to capture digital images. The camera is either a camera port for plugging in an external camera, such as an intra-oral camera, or is wholly contained within the collimator tube. The radio frequency transmitter transmits digital data to a computer. The frame grabber captures digital data for display on a display device. The transilluminator light can illuminate an area for visual inspection and/or digital capture. A receptor holder includes a docking port for docking a sensor that is used for digital x-ray image capture.

Referring to FIG. 1, one embodiment of the image capture device of the present disclosure is illustrated and indicated generally at 10. Image capture device 10 is operable to capture x-ray images. The image capture device 10 illustrated in FIG. 1 includes x-ray generator 12 and collimator tube 14. Collimator tube 14 serves as the means for focusing x-rays produced by the x-ray generator 12. Alternatively or additionally, collimator tube 14 also decreases scatter radiation and/or decreases absorbed radiation, thereby lowering the patient's x-ray dose. In one embodiment, collimator tube 14 has a square shape in cross section. Collimator tube 14 can also be rectangular, star, cross or round in cross section shape, as a few additional non-limiting examples.

In one embodiment, collimator tube 14 is fixed to x-ray generator 12 and cannot be removed. In another embodiment, collimator tube 14 is detachable from x-ray generator 12, such as by removing one or more screws or other securing means. Alternatively or additionally, collimator tube 14 can be a collimator tube of one shape that replaces a previously attached collimator tube of a different shape. One non-limiting example includes detaching a round-shaped collimator tube and replacing it with a rectangular-shaped collimator tube.

Collimator tube 14 has a tunnel 16 for emitting x-rays and a receptacle 30 for receiving a sliding bar of a receptor holder. Collimator tube 14 has an end 17 that serves one or more purposes. One purpose of end 17 is for coupling a receptor holder to collimator tube 14. Collimator tube 14 has one or more devices integrated within walls 18. In one embodiment, at least one integrated device is visible at least in part from end 17. In one embodiment, collimator tube 14 has a radio frequency transmitter 20, a camera/port 22, a frame grabber 24, and/or a transilluminator light 26 integrated within one or more of walls 18.

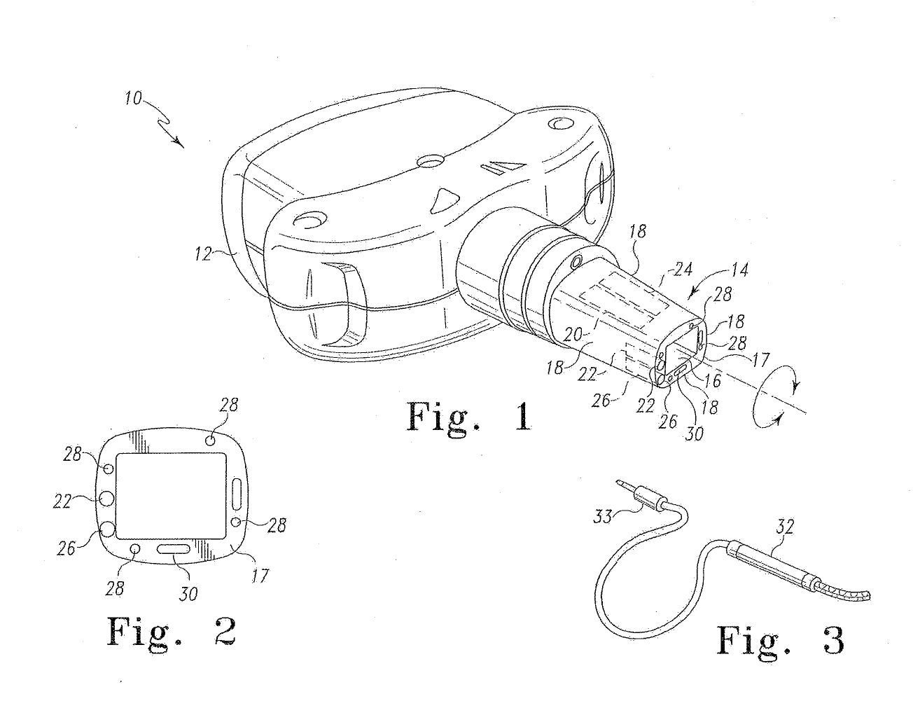

Each of these integrated devices will now be described in further detail with reference to FIGS. 1 and 2. Radio frequency (RF) transmitter 20 is operative to send data captured with image capture device 10 to an RF receiver external to image capture device 10. For example, the RF receiver may be coupled to a remotely-located computer for the display and/or processing of images captured by the device 10. As one non-limiting example, RF transmitter 20 can transmit data captured with camera 22 to an external computer. In this way, images captured by image capture device 10 may be downloaded to a computer system for current and/or later use without the need to physically couple the image capture device 10 to the computer. This greatly improves the maneuverability and usefulness of the image capture device 10.

In one embodiment, the camera 22 is built into the collimator tube 14 and is able to capture images visible from a lens or aperture formed into the end of the collimator tube 14. In another embodiment, camera 22 is an electrical port for allowing an external camera device to plug into collimator tube 14 for transmission of data from the external camera device to the collimator tube 14 integrated devices. One non-limiting example of an external camera device includes intra-oral camera 32 as shown in FIG. 3. The intra-oral camera 32 includes an electrical plug 33 that interfaces with the camera 22 port in the collimator tube 14 for transmitting data captured by the camera 32. Cameras 22 and 32 are operative to capture still images and/or video images in various embodiments. Provision of the external intra-oral camera 32 allows for easy access to the inner regions of the patient's mouth.

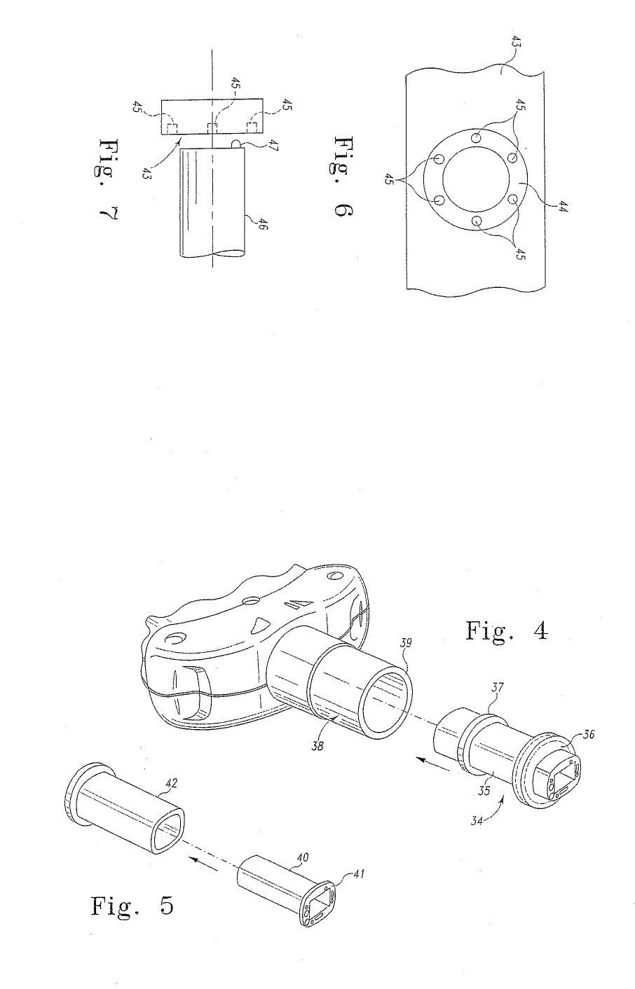

In one embodiment, frame grabber 24 is operative to capture still and/or video images for display on an external video display device, such as on a television or a computer display, as is known in the art. Frame grabber 24 is integrally formed with collimator tube 14 to receive image information from one of the image devices integrated with image capture device 10, such as camera 22/32 or the x-ray image receptor 44 (see FIG. 4). The output of frame grabber 24 is preferably coupled to the RF transmitter 20 for transmission of the frame data to a receiving computer and/or display device.

Transilluminator light 26 aids the image capture process by allowing for a light to be shined through a tooth, body or organ, as a few non-limiting examples. The light that is transmitted through the tooth can then be captured using the camera 22/32. For use with the camera 22 formed integrally with the collimator tube 14, means must be provided for directing the light from the transilluminator light 26 to the opposite side of the tooth as the camera 22. This may be done by use of an appropriate mirror (not shown), or by making the transilluminator light 26 an external device that plugs into a port in the collimator tube 14, similar to the intra-oral camera 32. Additionally, the transilluminator light 26 may emit light from the collimator tube 14 and the light transmitted through the tooth may be captured using the intra-oral camera 32.

Various other device combinations are also possible, such as fewer or additional devices than described herein, or a combination of those described. Power may be supplied to these devices by using the internal power supply of the image capture device 10, as will be apparent to those skilled in the art after reference to the above description.

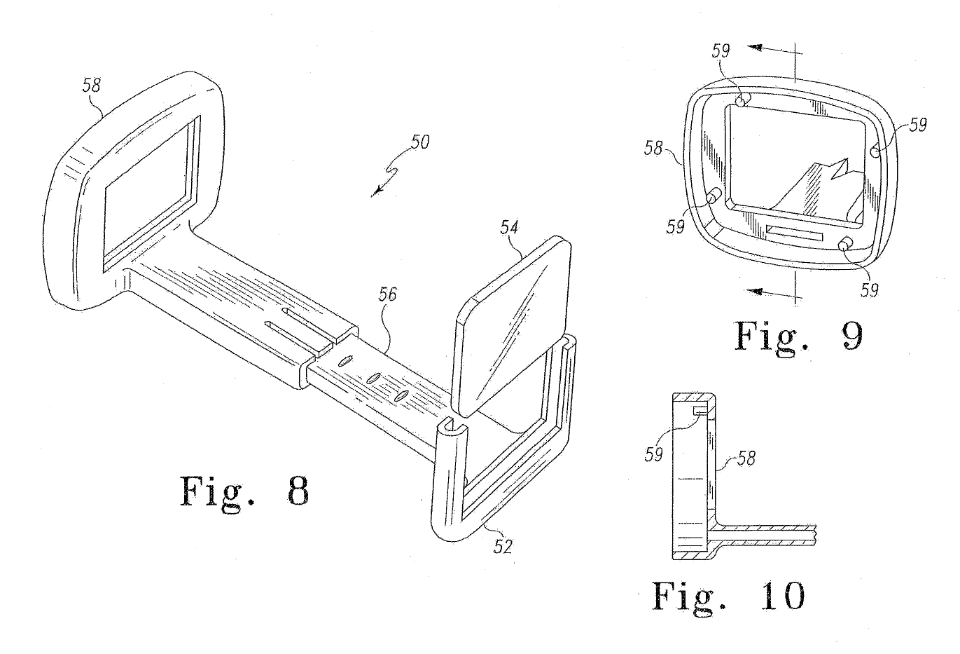

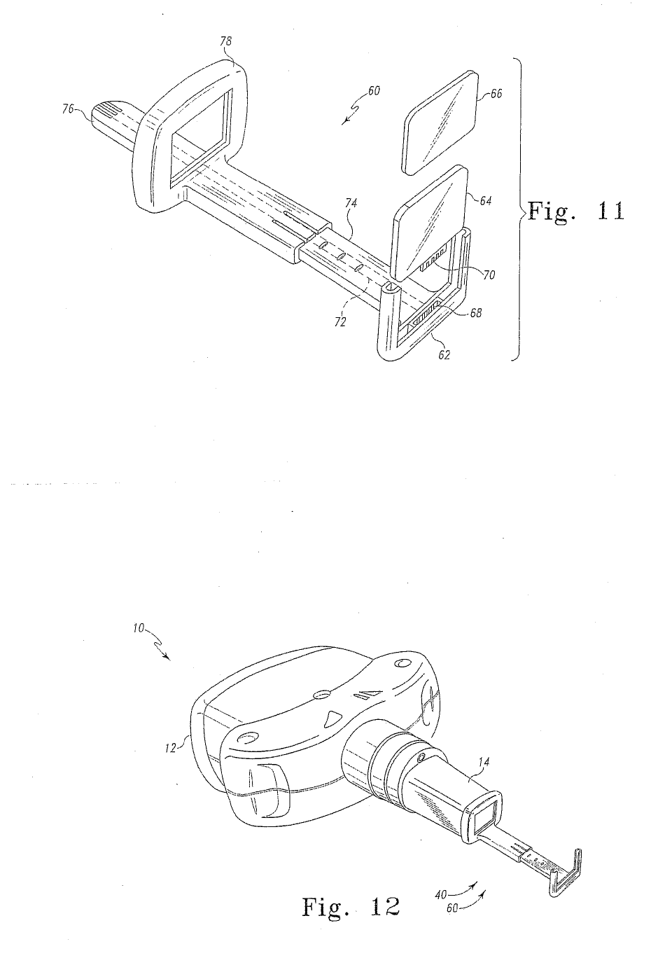

In one form, collimator tube 14 has contact sensor receptacles 28 that are used to mate an image receptor holder (see FIGS. 8-10) to the collimator tube 14. Contact sensor receptacle 28 can be one or more of various types, such as electrical, mechanical, optical, fiber optic, magnetic or of other connection types as would occur to one in the art. As one non-limiting example, contact sensor receptacles can be used to form a purely mechanical connection between the image receptor holder and the collimator tube 14. Alternatively or additionally, contact sensor receptacles 28 can be used to ensure a receptor holder is attached before firing x-ray generator 12. Alternatively or additionally, one or more lights can be illuminated to indicate the status of the connection, such as green to indicate a proper connection with collimator tube 14 has been made and the x-ray generator is ready to fire, and red to indicate the x-ray generator is not ready to fire, to name a few examples. Alternatively or additionally, a light can be illuminated to indicate that a proper connection has been made, and the light is not illuminated when a proper connection is not made. Alternatively or additionally, an audible sound can be emitted to indicate that a proper connection has been made. In one embodiment, contact sensor receptacles 28 are used with receptor holder 50 illustrated in FIGS. 8-10. Alternatively or additionally, collimator tube 14 has a contact end receptacle 30 formed therein and operative to receive a contact end of a receptor holder. In one embodiment, contact end receptacle 30 is used with the receptor holder illustrated in FIG. 11.

In yet another embodiment, as illustrated in FIG. 4, collimator tube adapter 34 is used to convert collimator tube 38 to a different shape. As one non-limiting example, collimator tube adapter 34 is used to convert collimator tube 38 from a round shape to a rectangular or other shape. Collimator tube adapter 34 includes cone 35, cap 36, and spacer 37. Cap 36 can slide to adjust cone 35 to different depths so cone 35 can fit properly inside collimator tube 38. Various mechanisms can be used to lock cap 36 into a desired location on cone 35, such as using detents or a snap ring, to name a few non-limiting examples. Spacer 37 is used to help secure cone 35 inside collimator tube 38, since cone 35 is a different shape than collimator tube 38. Various types of spacers can be used, such as a washer or an o-ring, to name a few non-limiting examples. Cap 36 is operable to form a seal around end 39 of collimator tube 38.

Although not numbered on FIG. 4 to preserve clarity, in one embodiment, collimator tube adapter 34 has one or more devices integrated within its walls. These devices can be integrated within walls of collimator tube adapter 34 instead of or in addition to devices integrated in collimator tube 38. Collimator tube adapter 34 and collimator tube 38, when used together, can include the same devices and perform the same functions as described herein with respect to collimator tube 14 of image capture device 10 on FIG. 1.

As shown in FIG. 5, insert 40 is a step-down insert that can be inserted into collimator tube 42 to make the image capture area smaller. Alternatively or additionally, insert 40 can be used to add integrated devices to existing collimator tube 42. One non-limiting example of a situation in which insert 40 can be used is to capture an image more precisely on a smaller image receptor than collimator tube 42 would capture alone. Alternatively or additionally, insert 40 can be inserted into collimator tube adapter 34 of FIG. 4 to make the image capture area even smaller. Although not numbered on FIG. 5 to preserve clarity, instead of or in addition to the integrated devices included within the walls of collimator tube 14 or collimator tube adapter 34, insert 40 can optionally include one or more devices integrated within its walls. Insert 40 can optionally have at least one integrated device visible at least in part from end 41. Details about these integrated devices and how they function are described in detail in reference to FIGS. 1 and 4. Alternatively or additionally, insert 40 can be used to add one or more integrated devices to existing collimator tube 42 without reducing the size of the image capture area any more than necessary to house the integrated devices.

As shown in FIG. 6-7, in one embodiment, a collimator tube and/or x-ray generator can rotate with respect to the other. Front end of x-ray generator 43 can optionally include a circular or other path 44 with indentations 45. Collimator tube 46 mates with front end of x-ray generator 43 with one or more detents 47 that lock and unlock into one or more of indentations 45 when rotated along path 44. Circular or other path 44 can be a track or other types as would occur to one of ordinary skill in the art so as to allow collimator tube 46 inserted therein to remain physically attached to the front end of x-ray generator 43 and then click into position when coming into contact with one or more detents 47. In an alternative embodiment, the indentations and path are present on collimator tube 46 and one or more detents are present on front end of x-ray generator 43.

Referring now to FIGS. 8-10 with continued reference to FIGS. 1-2, a first embodiment receptor holder 50 is illustrated. Receptor holder 50 has a holder end 52 that is operative to hold an image receptor 54, such as x-ray film or a digital charge-coupled device (CCD) sensor, as a few non-limiting examples. Receptor holder 50 has an adjustable bar 56 for adjusting the distance of holder end 52 from a connection end 58. In one embodiment, connection end 48 has a square shape. Connection end 58 can also be rectangular, star, cross or round in cross section shape, as a few additional non-limiting examples.

As shown in FIGS. 9 and 10, connection end 58 may have contact sensors 59 that are operative to be connected with collimator tube 14 through contact end receptors 28 formed in collimator tube end 17. In one embodiment, contact sensors 59 are used to ensure that the receptor holder 50 is attached to collimator tube 14 before firing the x-ray generator 12. In this embodiment, contact end receptors 28 are formed with sensors that determine when contact sensors 59 are inserted therein. As one example, each receptor 28 may have two metallic elements that are short circuited by a conductive contact sensor 59 when the connection end 58 is fitted to the collimator tube end 17. Coupling these metallic elements to the firing circuitry of the image capture device 10 can prevent the image capture device 10 from being fired unless the receptor holder 50 is properly fitted, as will be apparent to those skilled in the art from the above description. In one embodiment, receptor holder 50 is used to capture film-based x-ray images. In another embodiment, receptor holder is used to capture digitized x-ray images. Alternatively or additionally, receptor holder 50 can be used to hold a mirror for reflecting an image to be captured with camera 22, or for reflecting transilluminator light 26 for capture with camera 22.

Referring now to FIG. 11 with continued reference to FIGS. 1-2, a second embodiment receptor holder 60 is illustrated. Receptor holder 60 has a holder end 62 that is operative to hold a digital sensor 64 or x-ray film 66. Alternatively or additionally, receptor holder 60 can be used to hold a mirror for use with camera 22 or transilluminator light 26, as described hereinabove. Holder end 62 contains a docking port 68 to enable connection of digital sensor 64, which includes a sensor end 70 for electrical mating to docking port 68 of holder end 62. In one embodiment, wiring 72 is connected to docking port 68 at holder end 62. Wiring 72 runs through adjustable bar 74 and connects to contact end 76. Receptor holder 60 has adjustable bar 74 for adjusting the distance of holder end 62 from contact end 76. In one embodiment, adjustable bar 74 can slide all the way out and separate from connection frame 78. Contact end 76 can be inserted into contact end receptacle 30 of collimator tube 14 to couple receptor holder 60 to collimator tube 14.

Upon insertion of contact end 76 into contact end receptacle 30, connection frame 78 fits over end 17 of collimator tube 14. In one embodiment, when digital sensor 64 is docked in docking port 68 and x-ray generator 12 is fired, digital data is captured using sensor 64 and travels through wiring 72 to contact end 76 and to frame grabber 24 of collimator tube 14. In another embodiment, digital data captured using sensor 64 travels through wiring 72 to contact end 76 and to radio frequency transmitter 20 of collimator tube 14. In this embodiment, a frame grabber may be present in the remote computer.

In an alternate embodiment, receptor holder 50 (FIG. 8) and/or receptor holder 60 (FIG. 11) has an integrated radio frequency transmitter, such as within the walls of the holder.

FIG. 12 illustrates an embodiment of the disclosed system comprising image capture device 10 with x-ray generator 12, collimator tube 14, and receptor holder (50 or 60) attached.

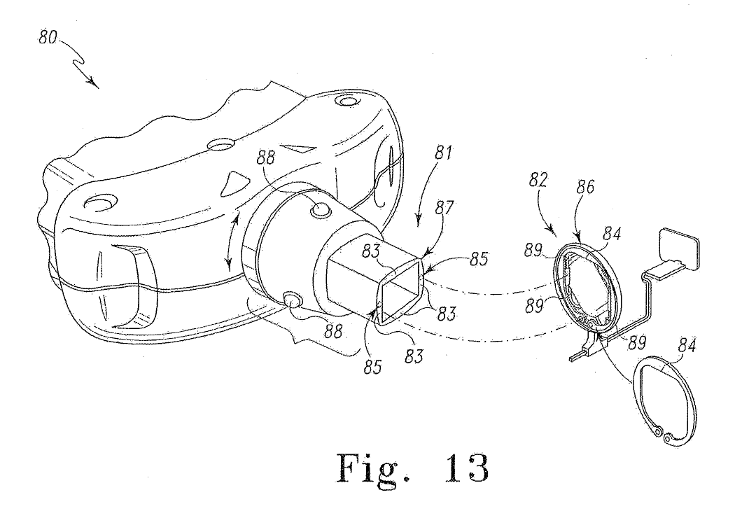

FIG. 13 illustrates an image capture device having an x-ray generator 80, collimator tube 81, and receptor holder 82. Film alignment ring 86 of receptor holder 82 fits around outer perimeter 87 of collimator tube 81. In one embodiment, notches 89 in receptor holder 82 are tapered and/or indented to help film alignment ring 86 align properly with collimator tube 81. Receptor holder 82 can be coupled to collimator tube 81 by magnetism when magnets 83 of collimator tube 81 come into contact with conductor/magnet interface metal snap ring 84 of receptor holder 82. Alternatively or additionally, snap ring 84 completes the circuit between low voltage electrical contact 85 and magnets 83, causing LED lights 88 to illuminate to indicate that receptor holder 82 is properly coupled to collimator tube 81. Magnets 83 and/or electrical contacts 85 can be located at one or more of various locations on outer perimeter 87 or other locations as would occur to one of ordinary skill in the art. In one embodiment, collimator tube 81 is a replacement for a prior collimator tube of a differing shape, such as a rectangular tube replacing a round tube, to name a non-limiting example. Alternatively or additionally, magnets 83, electrical contacts 85, and LED lights 88 can be included in an adaptor that is attached to an existing collimator tube. In one embodiment, receptor holder 82 and collimator tube 81 maintain a magnetic connection sufficient to couple them together but to also to allow them to easily separate from each other upon contact, such as with a doctor or patient touching receptor holder 82. Alternatively or additionally, the positioning bar of receptor holder 82 has notches that allow the user to identify the distance at which the film was positioned, such as to allow for re-taking another image in the future at the same distance.

Although not shown on FIG. 13 to preserve clarity, in one embodiment, collimator tube 81 has one or more devices integrated within its walls. Collimator tube 81 can include the same devices and perform the same functions as described herein with respect to collimator tube 14 of image capture device 10 on FIG. 1.

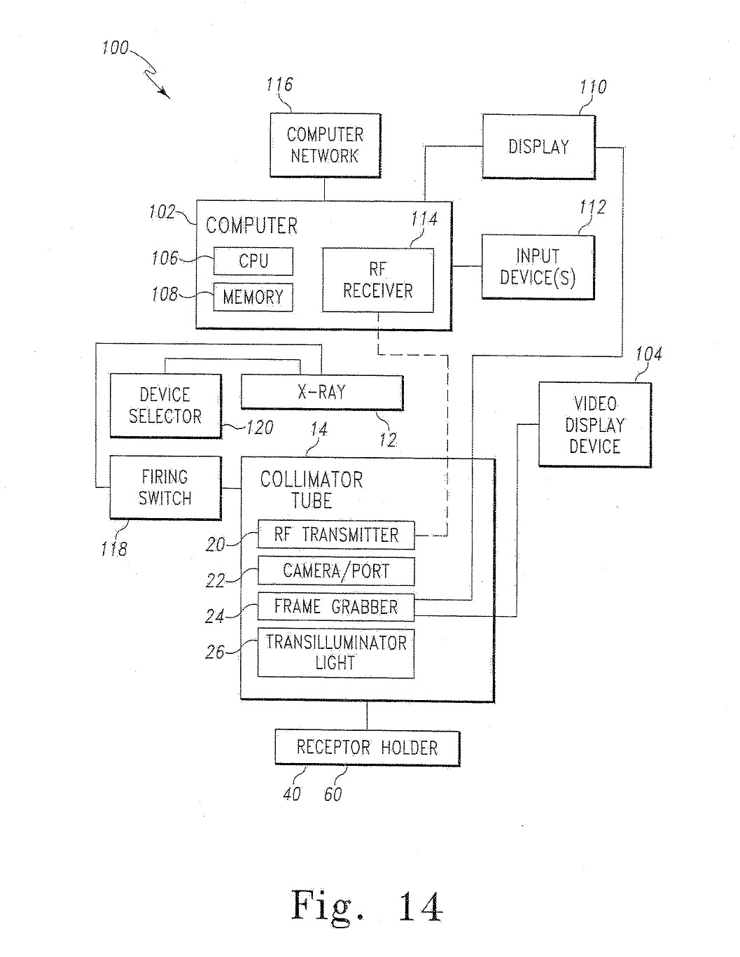

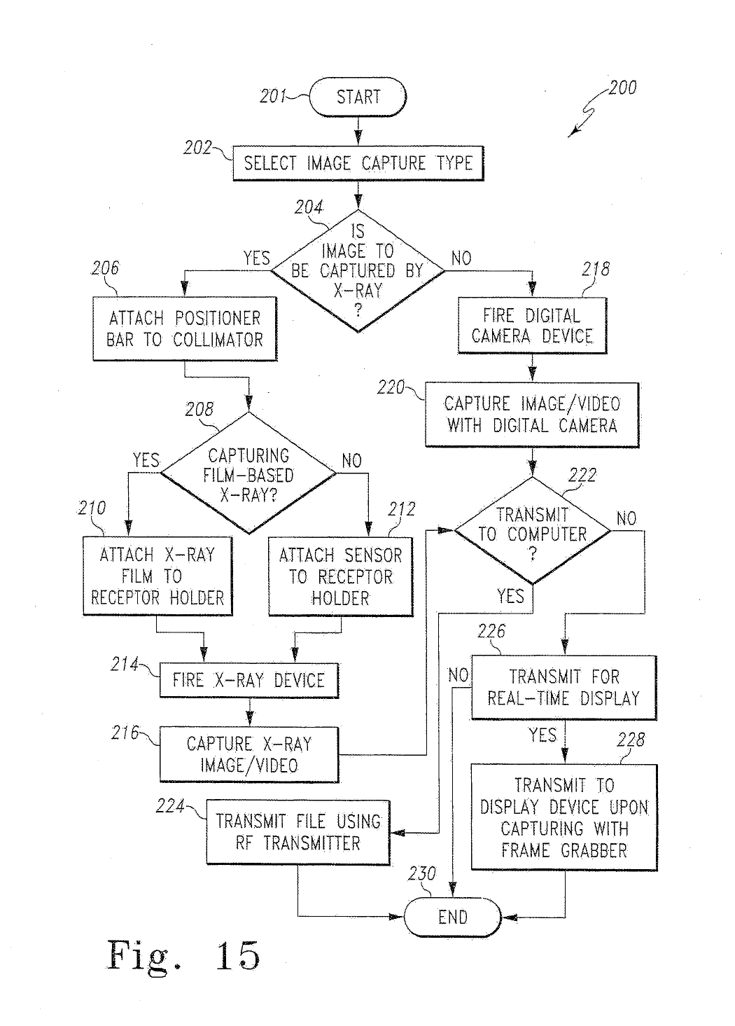

Reference will now be made to FIGS. 14 and 15 with continued reference to FIGS. 1-13 to illustrate a system and method for using image capture device 10. The same reference numerals are used to refer to elements that have already been introduced. FIG. 14 is a diagrammatic view of system 100. System 100 includes computer 102, x-ray generator 12, collimator tube 14, receptor holder (50 or 60), and video display device 104. It should be understood computer 102 may be arranged to include both a client and server, just a client, or just a server. Furthermore, it should be understood that while one computer is illustrated, more than one computer may be utilized in alternative embodiments.

Computer 102 includes one or more processors or CPUs 106 and one or more types of memory 108. Each memory 108 may include a removable memory device, although not shown to preserve clarity. The processor 106 may be comprised of one or more components configured as a single unit. Alternatively, when of a multi-component form, a processor 106 may have one or more components located remotely relative to the others. One or more components of each processor 106 may be of the electronic variety defining digital circuitry, analog circuitry, or both. In one embodiment, processor 106 is of a conventional, integrated circuit microprocessor arrangement, such as one or more PENTIUM III or PENTIUM 4 processors supplied by INTEL Corporation of 2200 Mission College Boulevard, Santa Clara, Calif. 95052, USA.

Memory 108 (removable or generic) is one form of computer-readable device. Memory 108 may include one or more types of solid-state electronic memory, magnetic memory, or optical memory, just to name a few. By way of non-limiting example, memory 108 may include solid-state electronic Random Access Memory (RAM), Sequentially Accessible Memory (SAM) (such as the First-In, First-Out (FIFO) variety or the Last-In-First-Out (LIFO) variety), Programmable Read Only Memory (PROM), Electronically Programmable Read Only Memory (EPROM), or Electrically Erasable Programmable Read Only Memory (EEPROM); an optical disc memory (such as a DVD or CD ROM); a magnetically encoded hard disc, floppy disc, tape, or cartridge media; or a combination of any of these memory types. Also, memory 108 may be volatile, nonvolatile, or a hybrid combination of volatile and nonvolatile varieties.

Computer 102 includes a display 110 and one or more input devices 112. Input devices 112 may include one or more operator input devices such as a keyboard, electronic pen input device, mouse, track ball, light pen, to name just a few representative examples. Computer includes a radio frequency (RF) receiver 114 for receiving data transmitted by radio frequency transmitters. Alternatively or additionally, computer 102 includes a printer. In one embodiment, computer 102 is disconnected from computer network 116. In another embodiment, computer 102 is connected to network 116.

Although only one computer 102 is shown to preserve clarity, more computers could also be present. In such instances, multiple computers 102, displays 110, and input devices 112 may be of the same respective type, or a heterogeneous combination of different computing devices. When more computers are present, computer 102 can be coupled to other computers over computer network 116. Computer network 116 could be in the form of a Local Area Network (LAN), Municipal Area Network (MAN), Wide Area Network (WAN), such as the Internet, a combination of these, or such other network arrangement as would occur to those skilled in the art. The one or more features provided by computer 102 can be provided on the same computer or varying other arrangements of computers at one or more physical locations.

X-ray generator 12 is operable to generate x-ray images. Collimator tube 14 serves as the means for focusing x-rays for x-ray device 12, and also includes additional integrated devices. Alternatively or additionally, collimator tube 14 also decreases scatter radiation and/or decreases absorbed radiation, thereby lowering the patient's x-ray dose. In one embodiment, collimator tube 14 has a radio frequency (RF) transmitter 20 that can communicate with RF receiver 114 of computer 102. Alternatively or additionally, collimator tube has an integrated camera 22. In one embodiment, camera 22 is a camera port for allowing an external camera device to plug into collimator tube 14. One non-limiting example of an external camera device includes an intra-oral camera 32 of FIG. 3. In another embodiment, camera 22 is wholly contained within collimator tube 14.

Alternatively or additionally, collimator tube 14 includes frame grabber 24 for capturing of and transmission of still and/or video images to video display device 104 and/or to display 110 of computer 102. Frame grabber 24 may also transfer data to rf transmitter 20. In one embodiment, collimator tube 14 includes transilluminator light 26. Transilluminator light 26 allows for shining a light through a tooth, body or organ, to name a few non-limiting examples.

X-ray generator 12 and collimator tube 14 are coupled to firing switch 118 and device selector 120. In one embodiment, a single firing switch 118 is used to fire whatever device is selected by device selector 120. In another embodiment, each image capture device has its own firing switch 118, and thus device selector 120 is not used. In some embodiments, firing switch 118 comprises a pair of switches which must both be pressed to activate the chosen device.

The operating logic of system 100 can be embodied in signals in programming instructions, dedicated hardware, transmitted over computer network 116, or a combination of these.

As one non-limiting example, system 100 can be used by a dentist to capture patient images. The image capture apparatus, system and method of the current disclosure are not limited to use in dentistry, or the field of medicine, as will be understood by one in the art. The systems disclosed herein can be used in various industries where capturing an x-ray image or digital image would be useful.

Referring additionally to FIG. 15, one embodiment for implementation with system 100 is illustrated in flow chart form as procedure 200, which demonstrates a high level process flow diagram of some of the features provided by system 100. In one form, procedure 200 is at least partially implemented in the operating logic of system 100. Procedure 200 begins at start point 201 with selecting an image capture type (stage 202). In one embodiment, image capture type is selected using device selector 120.

If the image to be captured by image capture device 10 is an x-ray image (decision point 204), then a receptor holder (50 or 60) is attached to collimator tube 14 (stage 206). If the image is to be captured on x-ray film (decision point 208), then x-ray film is attached to receptor holder (50 or 60) (stage 210). If the image is to be captured using digital x-ray (decision point 208), then digital sensor 64 is attached to receptor holder 60 (stage 212). The x-ray generator 12 is then fired using firing switch 118 (stage 214) and the x-ray image(s) and/or video are captured (stage 216) by the film or the sensor 64.

If the image to be captured by image capture device 10 is not an x-ray image but instead is to be captured by digital camera (decision point 204), then digital camera 22 is fired using firing switch 118 (stage 218) and the digital image(s) and/or video are captured (stage 220).

If image(s) and/or video captured digitally with the x-ray generator 12 or the digital camera 22 are to be transmitted to a remote computer (decision point 222), then RF transmitter 20 sends the digital file(s) to RF receiver 114 of computer 102 (stage 224). If the image(s) and/or video captured digitally with the x-ray receptor 64 or the digital camera 22 are to be displayed in real-time (decision point 226), then frame grabber board 24 intercepts the images captured with sensor 64 or digital camera 22 accordingly and transmits them (using RF transmitter 20) to computer 102, video display device 104, or computer display 110 (stage 228). The process then ends at stage 230.

Turning now to FIG. 16, an image capture device 240 is shown. In one embodiment, image capture device 240 has the same or similar capabilities as described previously with respect to FIG. 13. Image capture device 240 has an x-ray generator 242, collimator tube 244, and receptor holder 246. Receptor holder 246 has connection end 247 that connects to collimator tube 244. Film alignment ring 248 of receptor holder 246 fits around outer perimeter 250 of collimator tube 244. Digital sensor 252 has a contact plug 254 that plugs into receptacle 256 in bite block 258. Bar 260 is removable and couples bite block 258 to alignment ring 248 through arm 264. In one embodiment, bar 260 is metal, although other variations are possible. Data captured with sensor 252 travels through bar 260, to alignment ring 248. When collimator tube 244 is coupled to alignment ring 248, such as through magnetism, digital data then travels through a conductive path of alignment ring 248 to the low voltage electrical contact(s) 262 of collimator tube.

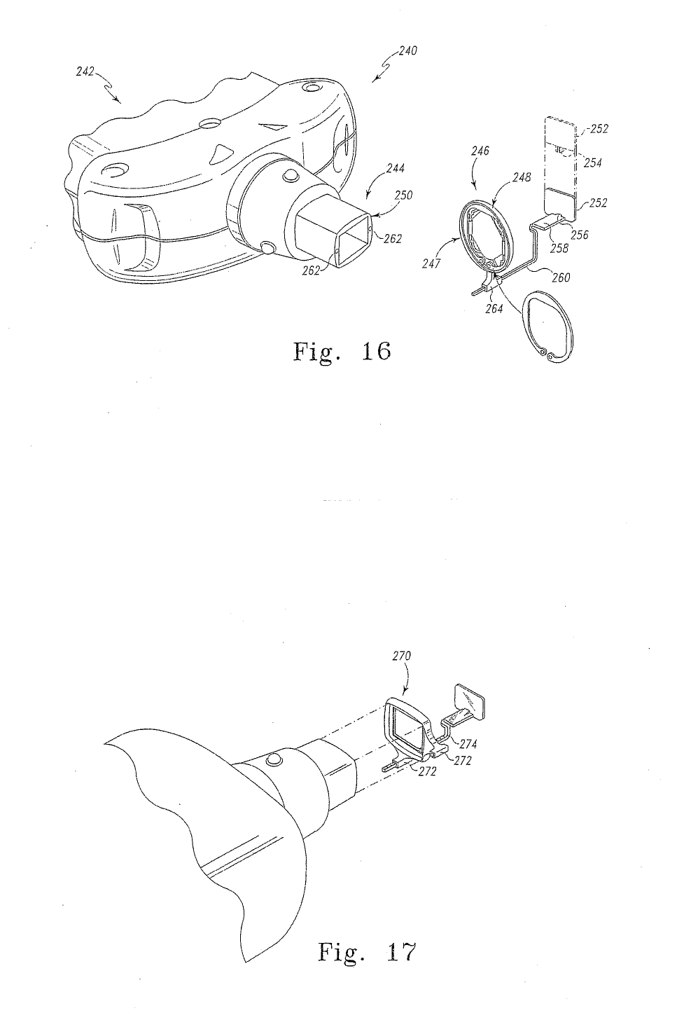

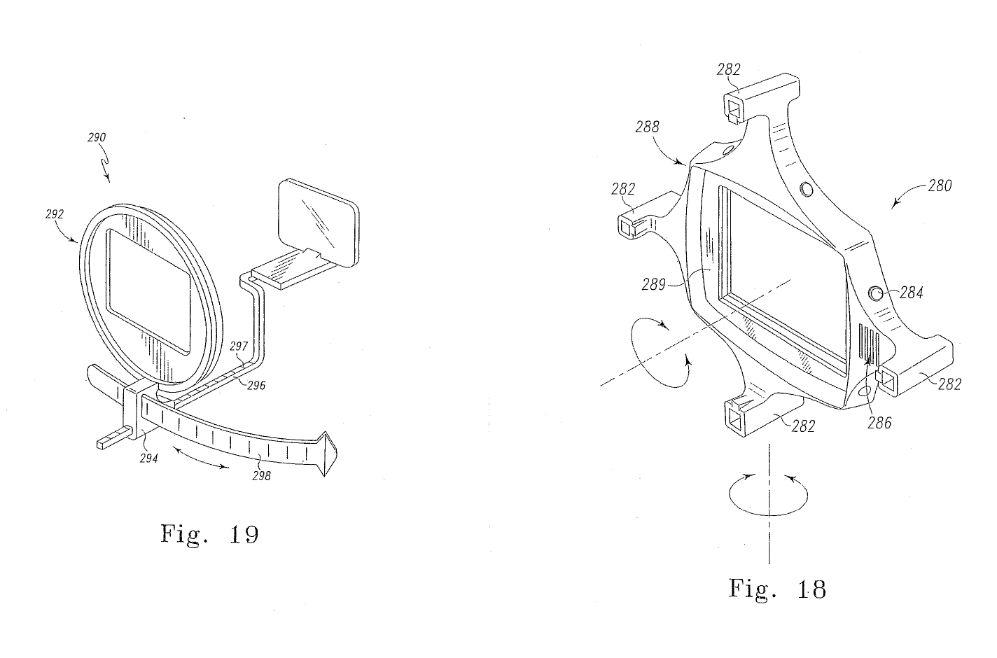

Turning now to FIG. 17, receptor holder 270 has multiple arms 272 for receiving bar 274. Arms 272 allow bar 274 to be positioned in one of a variety of positions for capturing different types of images. In one embodiment, receptor holder 270 has the same features as previously described with respect to receptor holder 246 of FIG. 16. Similarly, as shown on FIG. 18, receptor holder 280 includes multiple arms 282, namely four in the example illustrated. Arms 282 allow a bar inserted therein to be positioned in one of a variety of positions for capturing different types of images. In one embodiment, connection end 288 of receptor holder 280 includes a metal contact plate 289 for communicating with electrical contacts on a collimator tube. In one embodiment, receptor holder 280 has one or more lights, such as LED lights 284. In one embodiment, indicator lights, such as LED lights, illuminate when receptor holder 280 has mated properly with electrical contacts 262 (see FIG. 17). Alternatively or additionally, receptor holder 280 has a beeper device 286. In one embodiment, beeper device 286 produces a sound to indicate that receptor holder 280 has mated properly with electrical contacts 262 (see FIG. 17). In one embodiment, receptor holder 280 is reversible.

As shown in FIG. 19, receptor holder 290 includes a connection end 292 that can be coupled to a collimator tube. Connection end 292 includes at least one arm 294. Arm 294 allows a removable bar or other holder 296 to be coupled to connection end 292. Removable bar 296 includes notches or other markings 297 that allow a distance to be measured from a patient. Alternatively or additionally, receptor holder includes a measurement guide 298 which allows a distance to be measured from a patient. In one embodiment, measurement guide 298 is reversible, and can be used for left and/or right measurements. As one non-limiting example, markings 297 and/or measurement guide 298 can be used to measure the horizontal and/or vertical distance from a patient that a particular image is taken. Multiple measurements can be taken over a period of time if desired, such as to track the bone density of a particular patient.

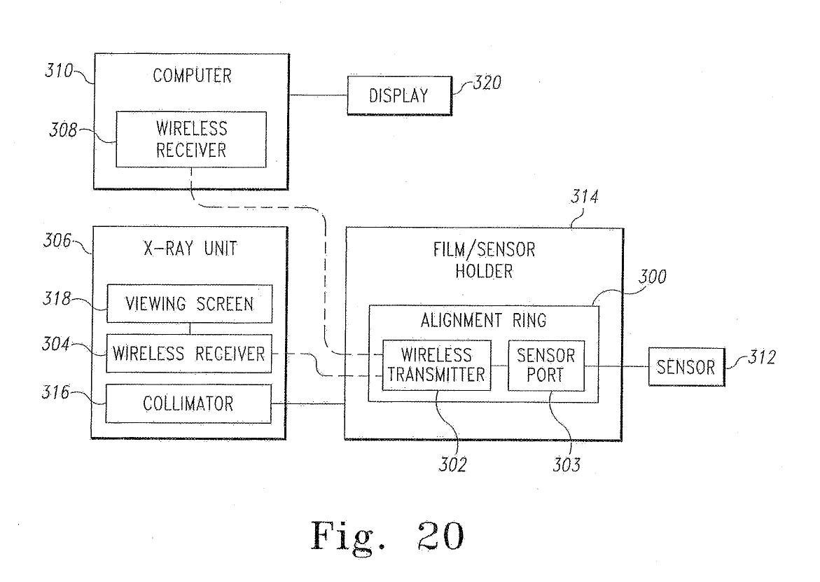

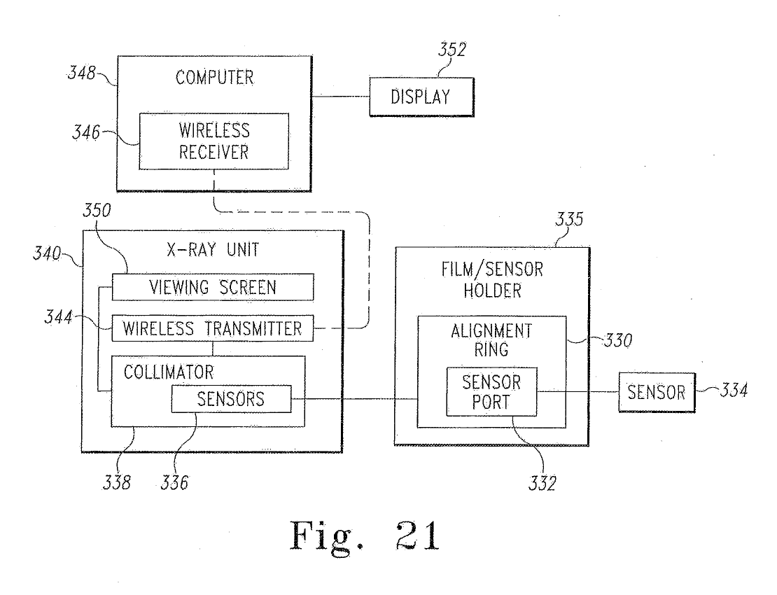

Turning now to FIGS. 20-26, various schematic system diagrams are shown that illustrate image capture systems having various components for capturing digital images in various ways. For example, in the system as shown in FIG. 20, an alignment ring 300 includes a wireless radio frequency transmitter 302 and a sensor port 303. One of ordinary skill in the art will appreciate that although radio frequency is used as the example wireless protocol with the examples throughout, various other wireless protocols could alternatively or additionally be used, such as infrared, to name one non-limiting example. Transmitter 302 is capable of transmitting data to the wireless receiver 304 of x-ray unit 306 and/or to the wireless receiver 308 of computer 310. Transmitter 302 is coupled to sensor port 303, and sensor port 303 is capable of receiving data from sensor 312. Alignment ring 300 can be coupled to receptor holder 314, and receptor holder can be coupled to collimator tube 316 of x-ray unit 306. When data is captured with sensor 312, it travels through sensor port 303, to wireless transmitter 302, and then to one or more of wireless receivers 304 or 308. Data can then be displayed on one of display screens 318 or 320. Data can alternatively or additionally be transmitted from transmitter 302 to other devices having a wireless radio frequency receiver.

In the system shown in FIG. 21, alignment ring 330 includes a sensor port 332. Sensor port 332 is capable of receiving data from sensor 334. Alignment ring 330 can be coupled to receptor holder 335 and then to sensors 336 of collimator tube 338 on x-ray unit 340. X-ray unit 340 includes a wireless radio frequency transmitter 344 that is capable of transmitting data to the wireless radio frequency receiver 346 of computer 348. Transmitter 344 is coupled to collimator tube 338. When data is captured with sensor 334, it travels through sensor port 332, to sensors 336 of collimator tube 338, and then to viewing screen 350, and/or to wireless transmitter 344 for transmission to receiver 346 for display on display device 352. Data can alternatively or additionally be transmitted from transmitter 344 to other devices having a wireless radio frequency receiver.

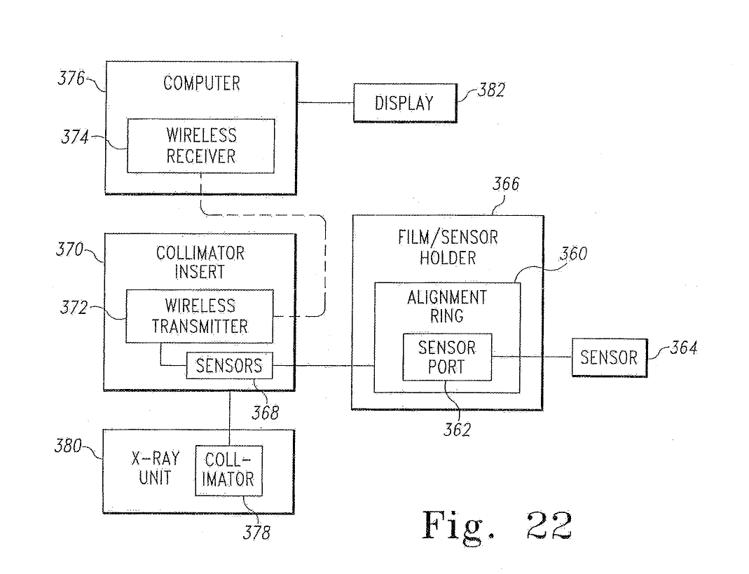

Turning now to FIG. 22, another image capture system is shown. An alignment ring 360 includes a sensor port 362. Sensor port 362 is capable of receiving data from sensor 364. Alignment ring 360 can be coupled to receptor holder 366 and then to sensors 368 of collimator tube insert 370. Collimator tube insert 370 can be coupled to an existing collimator tube 378 of x-ray unit 380. An example of collimator tube insert as described in FIG. 22 and some later figures is also described in reference to FIGS. 4 and 5. For example, insert 370 can be used to convert an existing collimator tube to a different shape, such as to convert a round collimator tube to a rectangular collimator tube. Alternatively or additionally, insert 370 can be used to allow digital devices to be added to an existing collimator tube of the same or different shape. Collimator tube insert 370 includes a wireless radio frequency transmitter 372 that is capable of transmitting data to the wireless radio frequency receiver 374 of computer 376. When data is captured with sensor 364, it travels through sensor port 362, to sensors 368 of collimator tube insert 370, and to wireless transmitter 372 for transmission to receiver 374 for display on display device 382. Data can alternatively or additionally be transmitted from transmitter 372 to other devices having a wireless radio frequency receiver.

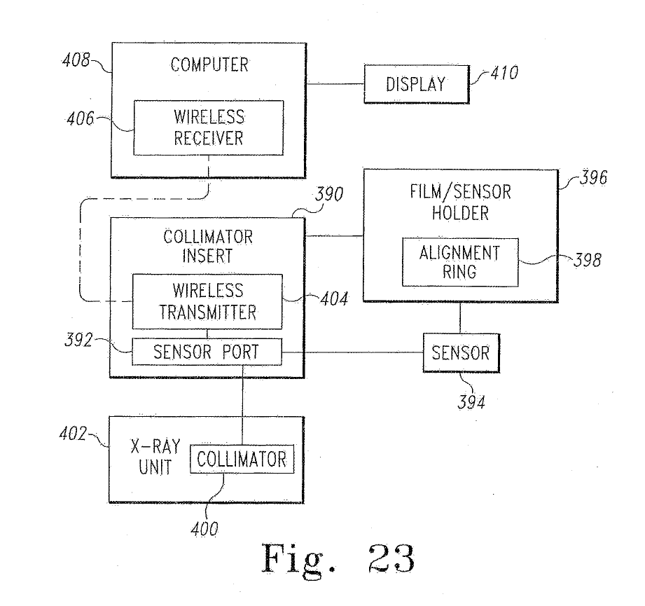

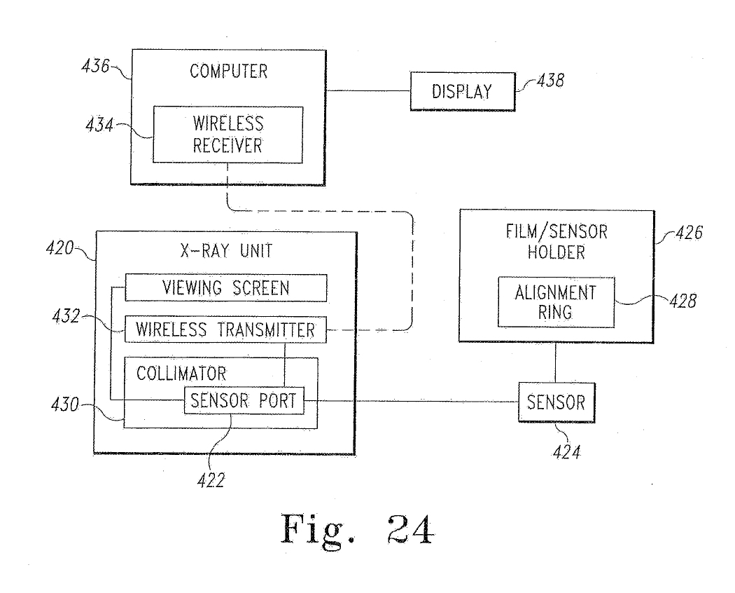

Turning now to FIG. 23, yet another image capture system is illustrated. Collimator tube insert 390 includes a sensor port 392. Sensor port 392 is capable of receiving data from sensor 394. Receptor holder 396 can be coupled to alignment ring 398. Sensor 394 can be coupled to receptor holder 396 to hold sensor 394 in a particular position. Receptor holder 396 can be coupled to collimator tube insert 390. Collimator tube insert 390 can be coupled to an existing collimator tube 400 of x-ray unit 402. Collimator tube insert 390 includes a wireless radio frequency transmitter 404 that is capable of transmitting data to the wireless radio frequency receiver 406 of computer 408. When data is captured with sensor 394, it travels through sensor port 392, and to wireless transmitter 404 for transmission to receiver 406 for display on display device 410. Data can alternatively or additionally be transmitted from transmitter 404 to other devices having a wireless radio frequency receiver.

As shown in FIG. 24, another image capture system is illustrated. X-ray unit 420 includes a sensor port 422. Sensor port 422 is capable of receiving data from sensor 424. Receptor holder 426 can be coupled to alignment ring 428. Sensor 424 can be coupled to receptor holder 426 to hold sensor 424 in a particular position. Receptor holder 426 can be coupled to collimator tube 430 of x-ray unit 420. X-ray unit 420 includes a wireless radio frequency transmitter 432 that is capable of transmitting data to the wireless radio frequency receiver 434 of computer 436. When data is captured with sensor 424, it travels through sensor port 422, and to wireless transmitter 432 for transmission to receiver 434 for display on display device 438. Data can alternatively or additionally be transmitted from transmitter 432 to other devices having a wireless radio frequency receiver.

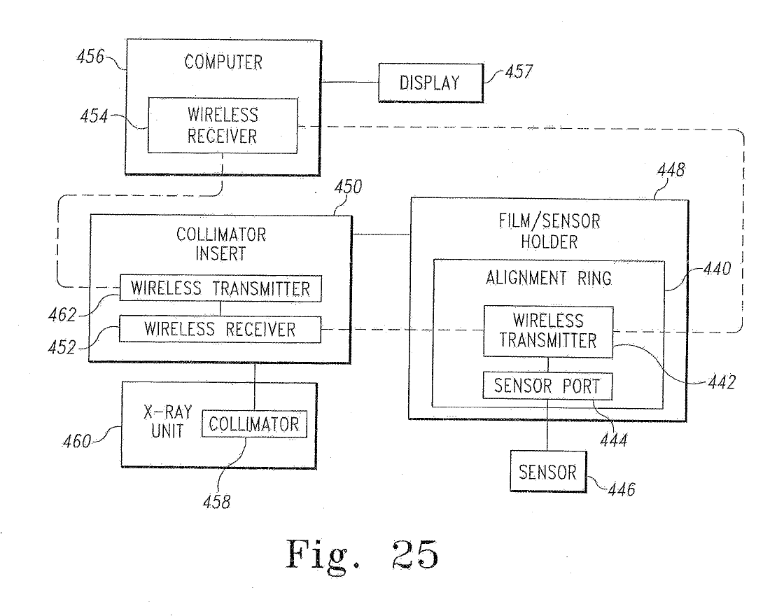

Turning now to FIG. 25, yet another image capture system is illustrated. Alignment ring 440 includes a wireless radio frequency transmitter 442 and a sensor port 444. Sensor port 444 is capable of receiving data from sensor 446. Receptor holder 448 can be coupled to alignment ring 440. Sensor 446 can be coupled to receptor holder 448 to hold sensor 446 in a particular position. Receptor holder 448 can be coupled to collimator tube insert 450. Collimator tube insert 450 can be coupled to an existing collimator tube 458 of x-ray unit 460. Wireless transmitter 442 of alignment ring 440 is operable to transmit data to wireless receiver 452 of collimator tube insert 450, and/or transmits data to wireless receiver 454 of computer 456 for display on display device 457. Wireless transmitter 462 of collimator tube insert 450 is operable to transmit data to wireless receiver 454 of computer 456 for display on display device 457. When data is captured with sensor 446, it travels through sensor port 444, to wireless transmitter 442, to wireless receiver 452, to wireless transmitter 462, and then to wireless receiver 454 for display on display device 457. Data can alternatively or additionally be transmitted from transmitter 442 and/or 462 to other devices having a wireless radio frequency receiver.

Turning now to FIG. 26, an image capture system having a portable sensor unit is illustrated. Portable sensor unit 470 includes a wireless radio frequency transmitter 472 and a sensor port 474. Sensor port 474 is capable of receiving data from sensor 476. Sensor 476 can be coupled to receptor holder 478 to hold sensor 476 in a particular position. Receptor holder 478 can be coupled to x-ray unit 480. Wireless transmitter 472 of portable sensor unit 470 is operable to transmit data to wireless receiver 482 of x-ray unit 480, to wireless receiver 484 of computer 486, and/or to wireless receiver 488 of another device 490. Data transmitted to wireless receiver 484 of computer 486 can be displayed on display device 492. When data is captured with sensor 476, it travels through sensor port 474, to wireless transmitter 472, and then to one or more of receivers 482, 484, or 488. Data can alternatively or additionally be transmitted from transmitter 472 to other devices having a wireless radio frequency receiver.

One of ordinary skill in the art will appreciate that the systems and devices described in FIGS. 20-26 can be used with the image capture devices, collimator tube inserts, receptor holders, and other novel features described in FIGS. 1-19. For the sake of simplicity, the detailed description of the devices already described in the prior figures were not repeated in reference to FIGS. 20-26. One of ordinary skill in the art will also appreciate that the systems and devices described in FIGS. 20-26 can also be used with other types of image capture devices that were not described in the prior figures.

In certain applications it is advantageous to provide systems and mechanisms for use with x-ray devices that provide enhanced delivery of care to patients by reducing exposure to x-radiation through close alignment of image receptors to the field of x-rays generated by x-ray devices. Certain embodiments of image receptors currently in use utilize lead backings to absorb x-radiation thereby reducing the amount passed on to patients. If image receptors are inadvertently or improperly placed outside of the field of x-rays, then less x-radiation is absorbed by the lead-backed receptors and more is passed on to patients. Improper placement may also result in unused area of the image receptor. Better use of the image receptor and correspondingly a reduction in patient exposure to x-radiation can be realized by more accurately positioning a lead backed image receptor within the field of x-rays. Better positioning can be accomplished through the use of alignment grids placed on or about the holder end of the receptor holder.

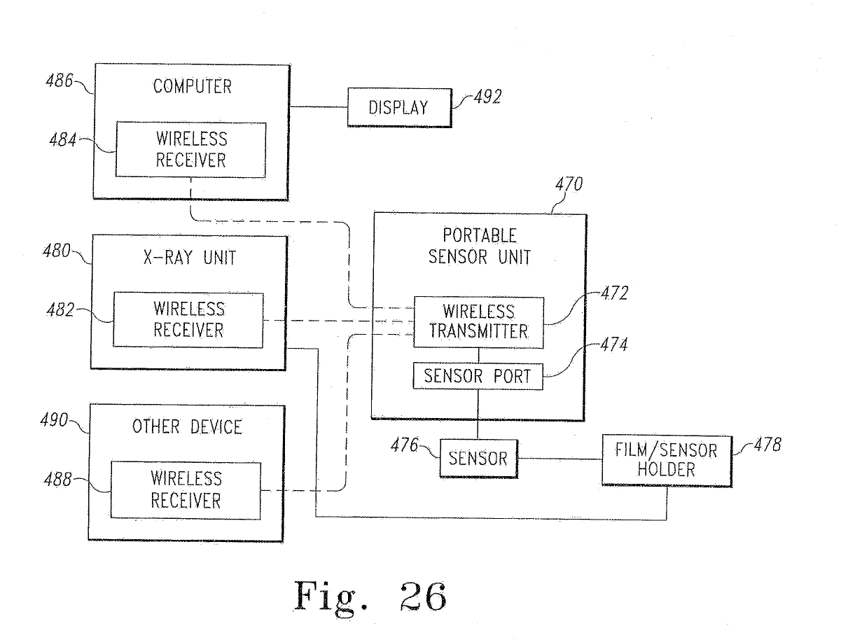

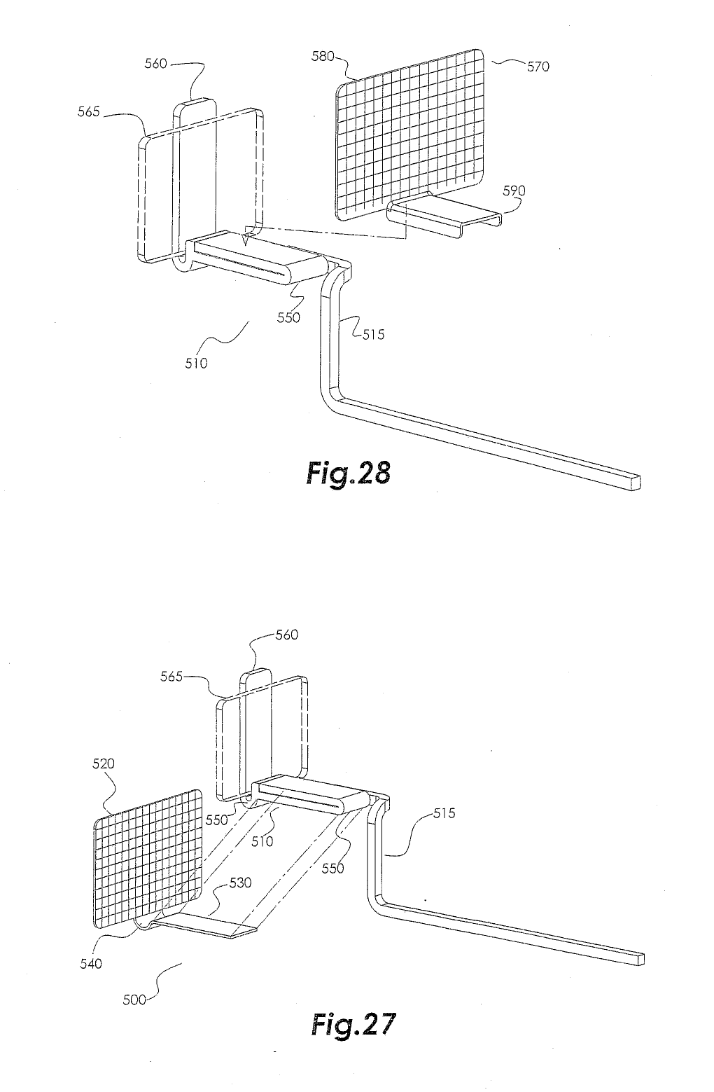

Turning now to FIG. 27, and referring generally to the holder end arrangements of FIGS. 13, 16, 17 and 19, gridded insert 500 is shown prior to placement on existing film holder device 510. Existing film holder device 510 is coupled to the x-ray device (not shown) using bar 515 (similar to bar 260 in FIG. 16). Gridded insert 500 has gridded part 520 and tongue 530 connected by arcuate member 540. Member 540 is adapted to fit in recess 550 formed in existing film holder device 510. Similarly, tongue 530 is adapted to be received by base 550, and gridded part 520 is adapted to be received by finger 560. When gridded insert 500 is received by existing film holder device 510, arcuate member 540 is adapted to receive x-ray film 565 such that gridded part 520 resides between finger 560 and x-ray film 565. Placement of gridded insert 500 behind x-ray film 565 allows a user of the x-ray device to align the edges of x-ray film 565 to the horizontal and vertical stripes shown in gridded part 520 thereby improving placement in the x-ray field and mitigating the patient's exposure to x-radiation. Gridded part 520 can be formed from a radiopaque material to further discourage transmission of x-radiation beyond the holder end and therefore reduce the patient's exposure to x-radiation. Such would be beneficial when used with an x-ray film that has no lead backing. In addition, gridded insert 500 can be made of material that can be sterilized to permit reuse of the insert from patient to patient. Gridded insert 500 can be adapted in some embodiments to be secured to film holder device 510 so as to prevent the movement of gridded insert 500 relative to the film holder device 510. Gridded insert 500 can be secured through the use of materials such as adhesive, for example. In other embodiments, gridded insert 500 can be configured without adhesive so as to promote rapid insertion and removal of gridded insert 500.

In another embodiment, FIG. 28 shows gridded insert 570 prior to placement on existing film holder device 510. As in FIG. 27, existing film holder device 510 is coupled to the x-ray device (not shown) using bar 515. Gridded insert 570 has gridded part 580 and mouth 590. Mouth 590 is adapted to be received by base 550. Unlike the insert shown in FIG. 27, gridded part 580 is not received by finger 560. Rather, x-ray film 565 is received by finger 560, and gridded part 580 is received by x-ray film 565 whereby x-ray film 565 resides between finger 560 and gridded part 580. The x-ray film 565 can be adjusted to align with the horizontal and vertical striping shown on gridded part 580 thereby improving placement in the x-ray field and mitigating the patient's exposure to x-radiation. It will be appreciated that gridded part 580 can be made of a clear radiotransparent material to allow passage of x-radiation in addition to providing proper alignment of x-ray film 565 prior to use. Gridded insert 570 can be removed from the holder end prior to insertion of film holder device 510 and x-ray film 565 into the patient's mouth to improve comfort by reducing the amount of device mass necessary when taking an x-ray image. Alternatively, gridded insert 570 can be secured to the image receptor and/or the film holder device 510 so as to prevent the movement of the gridded insert relative to the film holder device. Gridded insert 570 can be secured to the image receptor using materials such as adhesive, for example. In other embodiments, gridded insert 570 can be configured without adhesive so as to promote rapid insertion and removal of gridded insert 570. Gridded insert 570 can be made of material that can be sterilized to permit reuse of the insert from patient to patient.

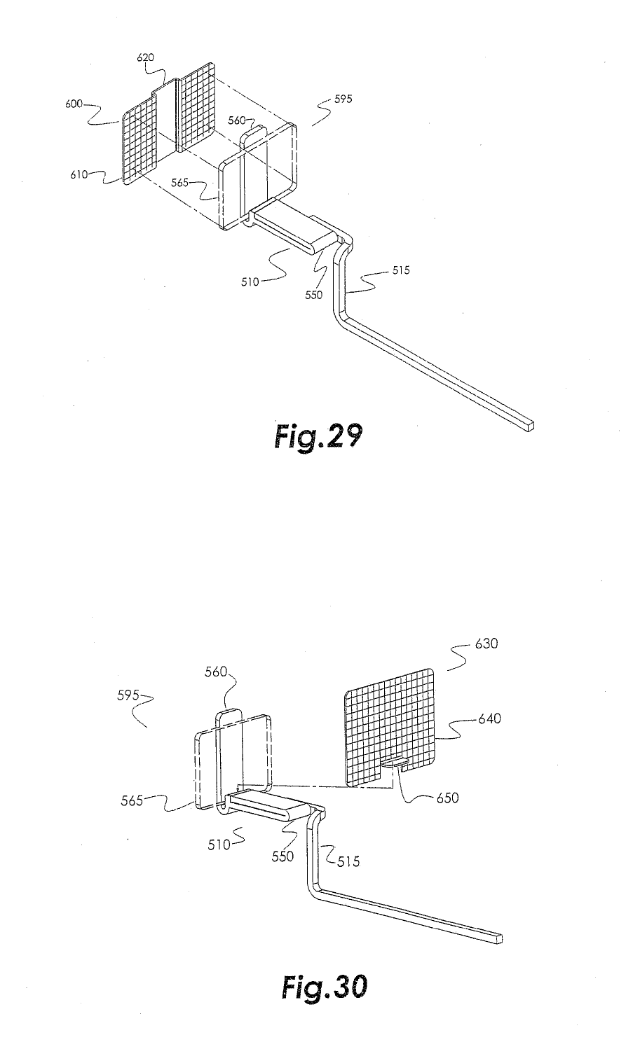

In a further embodiment, FIG. 29 shows gridded insert 600 prior to placement on existing film holder device 510. As in FIGS. 27 and 28, existing film holder device 510 is coupled to the x-ray device (not shown) using bar 515. Gridded insert 600 has gridded part 610 and slot 620. Slot 620 is adapted to be received by finger 560. Unlike the inserts shown in FIG. 27 and FIG. 28, gridded insert 600 is adapted to be received on the side of finger 560 opposite the base 550. When gridded insert 600 is in use, finger 560 resides between slot 620 of gridded insert 600 and x-ray film 565. Placement of gridded insert 600 behind x-ray film 565 allows a user of the x-ray device to visually align the edges of x-ray film 565 to the horizontal and vertical stripes shown in gridded part 520. Aligning the x-ray film using the gridded part can improve placement in the x-ray field to mitigate patient exposure to x-radiation. Gridded part 610 can be formed from a radiopaque material to further mitigate patient exposure to x-radiation. Such would be beneficial, for example, when used with an x-ray film that has no lead backing. In some applications, gridded insert 600 can be removed from the holder end prior to insertion in the patient's mouth so as to improve comfort by reducing the amount of device mass necessary when taking an x-ray image. In other applications, gridded insert 600 can be secured to finger 560 and/or image receptor 565 so as to prevent the movement of the gridded insert relative to the film holder device. Gridded insert 600 can be secured using materials such as adhesive, for example. In other embodiments, gridded insert 600 can be configured without adhesive so as to promote rapid insertion and removal of gridded insert 600. In these applications a secondary means of temporarily suspending gridded insert 600 is required, such as by use of a users fingers, for example. Gridded insert 600 can be made of material that can be sterilized to permit reuse of the insert from patient to patient.

In yet another embodiment, FIG. 30 shows gridded insert 630 prior to placement on existing film holder device 510. As in FIGS. 27, 28 and 29, existing film holder device 510 is coupled to the x-ray device (not shown) using bar 515. Gridded insert 630 has gridded part 640 and tab 650. Similar to mouth 590 in FIG. 28, tab 650 is adapted to be received by base 550. Finger 560, however, receives x-ray film 565 which itself receives gridded part 630 whereby x-ray film 565 resides between finger 560 and gridded part 630. The x-ray film 565 can be adjusted to align with the horizontal and vertical striping shown on gridded part 630 thereby improving placement in the x-ray field and mitigating patient exposure to x-radiation. It will be appreciated that gridded part 630 can be made of a clear radiotransparent material to allow passage of x-radiation in addition to providing proper visual alignment of x-ray film 565. Gridded insert 630 can be removed from the holder end prior to insertion in the patient's mouth to improve comfort by reducing the amount of device mass necessary when taking the x-ray. Alternatively, gridded insert 630 can be secured to the image receptor and/or the film holder device 510 so as to prevent the movement of the gridded insert relative to the film holder device. Gridded insert 630 can be secured to the image receptor and/or base 550 using materials such as adhesive, for example. In other embodiments, gridded insert 630 can be configured without adhesive so as to promote rapid insertion and removal of gridded insert 630. Gridded insert 630 can be made of material that can be sterilized to permit reuse of the insert from patient to patient.

In certain applications disclosed herein the use of alignment grids may increase the amount of materials present in the patient's mouth during use. It would be advantageous in some applications, therefore, to provide alternative systems and mechanisms that dispense with the need for alignment grids at the holder end of the receptor holder and instead provide for consistent placement of the image receptor within the field of x-rays generated by the x-ray device. The holder end can be configured having systems and mechanisms that provide for consistent insertion of the image receptor into the field of x-rays. Such would be desirable where the x-ray device is used with different patients and efficient and consistent replacement of the image receptor facilitates more rapid patient servicing.

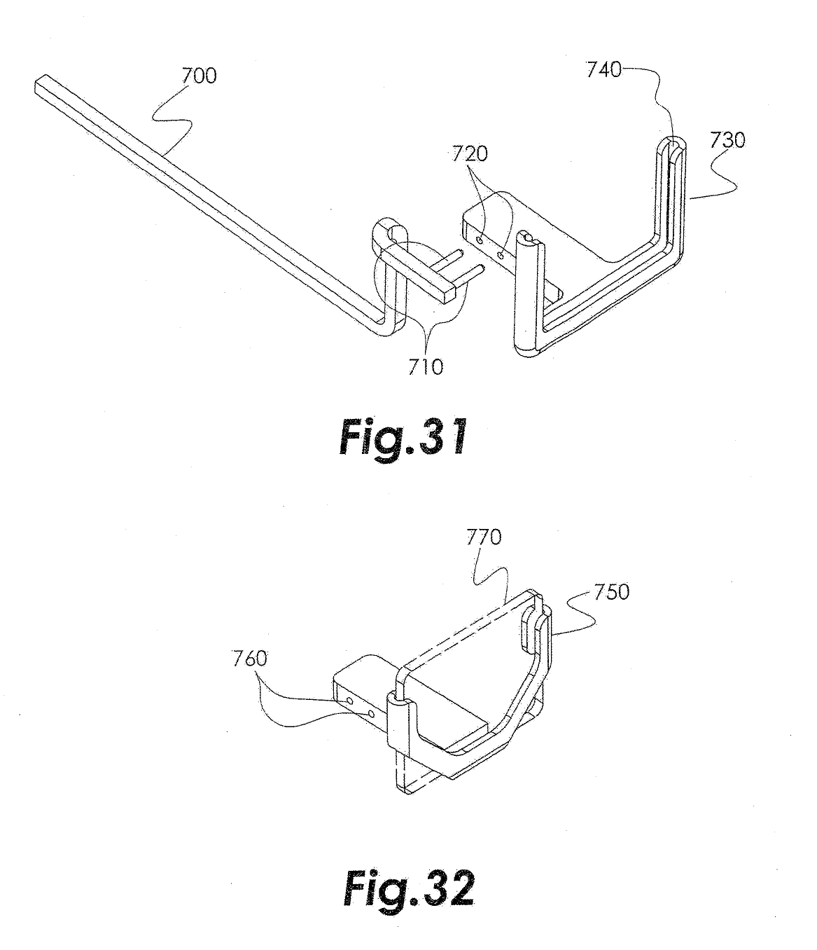

Turning now to FIG. 31, bar 700 is shown having pins 710 adapted to be received by holes 720 formed in an intermediate platform such as film holder device 730. Film holder device 730 is configured as three sided quadrilateral-shaped holder having generally U-shaped channels forming the three sides. The U-shaped channels are adapted to receive an image receptor and when in use the channels substantially enclose three sides of an image receptor. In some embodiments, film holder device 730 can be made of material that can be sterilized to permit reuse of the device from patient to patient. Alternatively and/or additionally, film holder device 730 can be made of a radiotransparent material to permit greater utilization of the image receptor. Film holder device can also be made of a radiopaque material to discourage transmission of x-radiation. In some embodiments, the U-shaped channels discourage movement of image receptors while in use. It will be appreciated that other embodiments of film holder device configurations are contemplated such as the configuration shown in FIG. 32.

Shown in FIG. 32 is film holder device 750 that is configured similar to quadrilateral shaped device 730 in FIG. 31 with the exception that the bottom corners have been chamfered thereby exposing all corners of x-ray film 770. Film holder devices 730 and 750 from FIGS. 31 and 32 respectively can be molded from any suitable material such as clear lexan, among others. It should also be noted that holes 760 are adapted to receive pins much as pins 710 on bar 700 in FIG. 31. In some embodiments, film holder device 750 can be made of material that can be sterilized to permit reuse of the device from patient to patient. Alternatively and/or additionally, film holder device 750 can be made of a radiotransparent material to permit greater utilization of the image receptor.

In some applications it would be advantageous to further reduce the amount of device mass within a patient's mouth by designing systems and mechanisms that dispense with the need for an intermediate platform such as a film holder device and instead design bars that have pins configured to interface directly with image receptors.

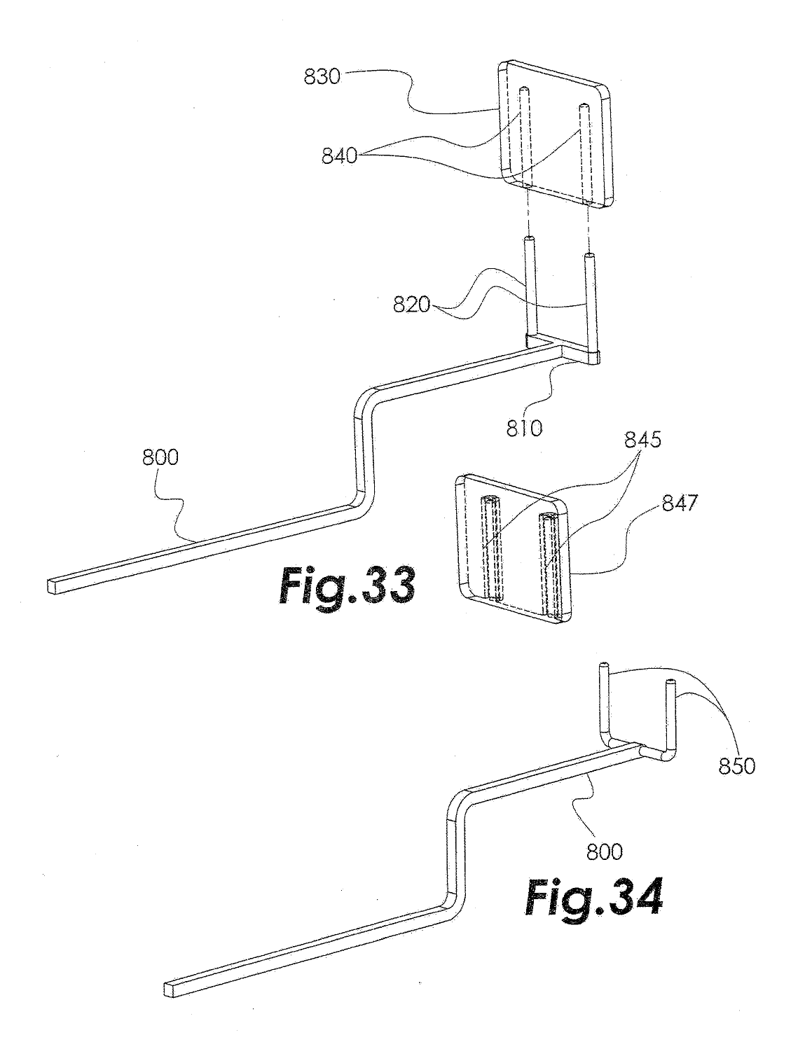

Turning now to FIG. 33, bar 800 is shown connected to cross member 810, which in turn is further connected to pins 820. Pins 820 are configured to be received by recesses 840 formed into image receptor 830. Pins 820 received in recesses 840 can remain attached through a press fit. Furthermore, pins 820 can resemble a right angle cylinder or can be tapered, among other geometric configurations. In one embodiment, recesses 840 can be molded into the casing of image receptor 830 so that the contour and form factor of the receptor appear similar to other recess-free receptors, such as those recess-free receptors that are enclosed in vinyl covering. In these embodiments the recesses are encapsulated in vinyl. In another embodiment, the recesses can also be molded into bumps 845 on the back of image receptor 847. It will be understood that other shapes and sizes of protuberances similar to bumps 845 are possible. In some embodiments, pins 820 can be made of a radiotransparent material to permit transmission of x-radiation through the pins. In other embodiments, pins 820 can be made of a radiopaque material. In still further embodiments, pins 820 can be adapted to be removable from cross member 810 to permit repair or refurbishing. Pins 820 can be made from a material that permits sterilization so as to promote reuse of the pins from patient to patient.

Through the use of the recesses described above it is possible for the receptors to be mounted directly to structure that extends from the bar rather than mounting to an intermediate platform such as a film holder device. The recesses can be used to permit receptors to be slidingly mounted to positions that are consistent from patient to patient. Such consistent positions and sliding arrangements can also shorten the time required of personnel to prepare for an x-ray image. Furthermore, in some embodiments the recess/mounting pin arrangement discourage cone-cutting and may also discourage bending of the image receptor relative to the base of the image receptor holder. Cone-cutting results when the x-radiation emitting from the collimation cone does not align in translation and/or rotation with the image receptor thus creating areas of the image receptor that are underutilized.

It can be seen that the structure shown in FIG. 33 utilizes three elements, that is, a cross member and two pins, that are connected to the bar. Structures similar to that shown in FIG. 33 that provide for mounting image receptors directly to bars can generally be referred to as mounting pin connections or mounting pin arrangements. It will be appreciated that an image receptor can be configured to adapt not only to the mounting pin arrangements as shown in FIG. 33 but also other types of pins such as shown in FIG. 31, for example. Other mounting pin arrangements to further arrange, or even reduce, the number of elements can be created. For example, the two pins 820 may be replaced by a single rectangular, triangular, or other geometrically shaped mounting structure that fits into a similarly shaped recess in image receptor 847.

FIG. 34, for example, shows an example of another type of mounting pin arrangement, this one using only two additional elements. Bar 800 is shown having mounting elements bent at right angles forming upturned pins 850. In this fashion only two elements are necessary to provide the necessary mounting pin arrangement required for mounting an image receptor. In some embodiments, pins 850 can be made of a radiotransparent material to permit transmission of x-radiation through the pins. In other embodiments, pins 850 can be made of a radiopaque material. In still further embodiments, pins 850 can be adapted to be removable from cross member 810 to permit repair or refurbishing. Pins 850 can be made from a material that permits sterilization so as to promote reuse of the pins from patient to patient. It will be appreciated that other types of mounting pin arrangements are possible for mounting image receptors. For example, mounting pin arrangements can be configured to provide for enhanced stability.

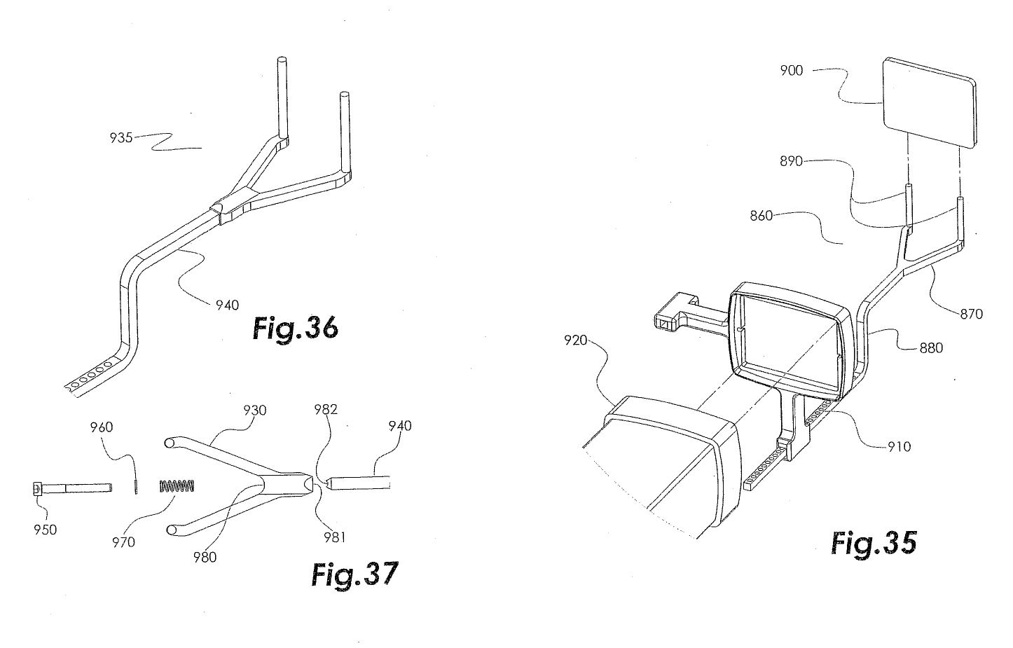

Turning now to FIG. 35, forked member 870 is shown attached to bar 880. Forked member 870 has two pins 890, which are adapted to receive an image receptor 900. Image receptor 900 can be adapted as described previously having internally molded recesses, recesses molded into external protuberances, or configured with adapters. Mounting pin arrangements like forked member 870 in some applications can provide for a more stable platform than the arrangements described above. When a patient bites across the two prongs of the fork the patient's teeth form a bridge that keeps the forked member 870 from rocking in the mouth therefore providing a stable platform on which to capture an x-ray image. In contrast, the single cross member on some of the arrangements described above can sometimes fail to keep the image receptor stable which may result in less than desirable image quality.

Bar 880 can be configured to have measurement indicators 910 to allow proper adjustment of image receptor 900 relative to collimator cone 920. Measurement indicators can be provided to permit a doctor or other medical personnel to capture images at consistent bar locations regardless of how distant in time the images are taken relative to one another. Images taken at consistent bar locations can be used to compare later acquired images to earlier images and in this way the progress of treatment can be better tracked. Measurement marks can also be useful in providing measurements for the so-called “subtraction radiation procedure,” a process allowing doctors to check bone density differences, for example when dental implants are being placed and monitored.

Forked member 870 can be formed in a variety of manners, one of which is to attach forked member 870 directly to bar 880. Forked member 870 can also be formed integral to bar 880. Other methods of formation and/or attachment are also possible.

Turning now to FIG. 36, forked member 935 is shown attached to bar 940 and is capable of rotation about bar 940. Such rotation permits image receptors mounted to forked member 935 to be rotated relative to the x-ray field therefore providing greater flexibility in the types and perspectives of images that can be captured in comparison to mounting pin arrangements that cannot be rotated. For example, vertical and horizontal images can be taken using the mounting pin arrangement in FIG. 36 simply by rotating forked member 935 from one position to the next. In some embodiments, forked member 930 can be rotated to a number of predetermined positions in much the same way a socket can be moved in a number of discrete steps around the head of a bolt. A rotating forked member similar to the illustrated embodiment allows a user to accomplish certain x-ray procedures with one device as opposed to many parts that may require assembly. As such, rotation mechanisms as disclosed in the illustrated embodiment permit a rotating head arrangement with relatively few parts.

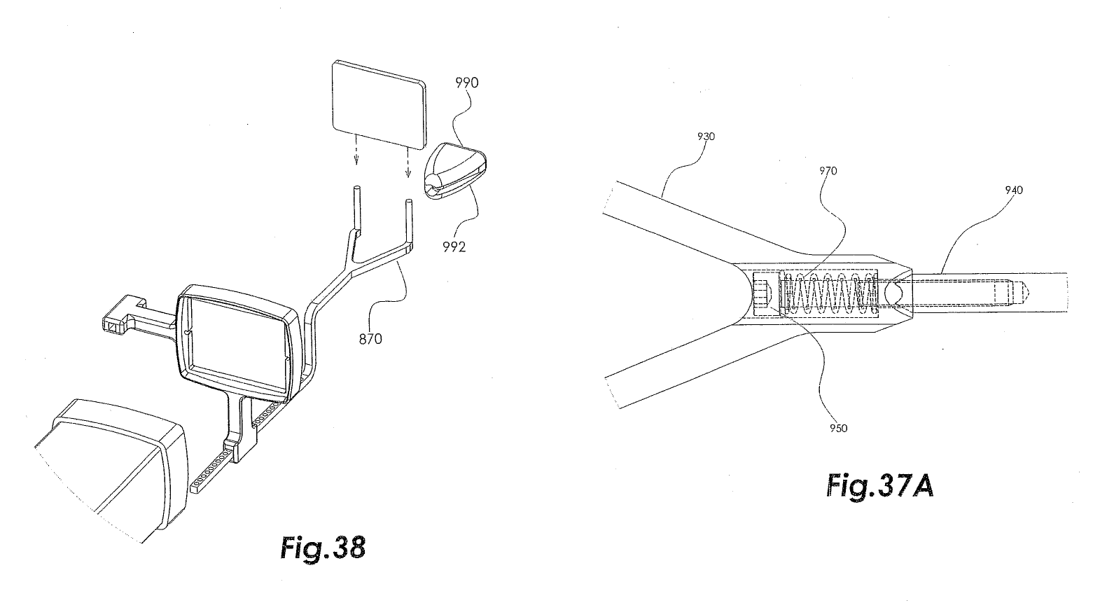

FIG. 37 shows an exploded view of forked member 930 and the attachment mechanisms that can be used to connect forked member 930 to bar 940 and create a number of preset positions. Bar 940 is adapted to receive forked member 930. Screw 950 can be inserted through washer 960, spring 970, valley 980 of forked member 930, and into bar aperture 982 of bar 940. Washer 960, screw 950, and spring 970 forms an attachment mechanism assembly.

With continuing reference to FIG. 37, FIG. 37a shows the attachment mechanism components as described in FIG. 37 operably installed creating a forked member capable of rotation about bar 940. The attachment mechanism assembly can be inserted into an aperture formed in valley 980 of forked member 930. The screw of the attachment mechanism assembly can then be inserted into bar aperture 982 and threaded into and therefore attached to bar 940. Washer 960 resides in receptor side aperture 980 of forked member 930. Spring 970 resides in the well of receptor side aperture 980. Spring 970 can be slightly compressed when installed and can urge forked member 930 in a direction toward bar 940. Forked member 930 contains a bar side aperture 981 that is adapted to receive bar 940. In this way, if bar 940 has a non-circular geometry, bar side aperture 981 is configured to receive the non-circular geometry. For example, if bar 940 were square-shaped, bar side aperture 981 would be configured to receive a square-shaped bar. The cooperative nature of the bar side aperture 981 and bar 940 create a mechanism similar to a socket connection.

When forked member 930 is desired to be rotated relative to bar 940, forked member 930 can be pulled away from bar 940 to release the socket connection mechanism. Forked member 930 can then be rotated freely about bar 940 to a newly desired location, at which point forked member 930 can be released and urged back toward bar 940 through the spring-action mechanism of spring 970. The number of desired positions is set by the number of sides present in the socket connection discussed above. The connections can include geometries such as triangle, square, pentagon, hexagon, among many others, and can also include other unique types of drive connections such as elliptically-based geometry and lobe engagements, some of which are sometimes referred to as TORX.

In some embodiments screw 950 can be a 2-56 UNC X ⅝″ socket cap screw, 18-8 stainless steel McM-C No. 92196A083 2287; washer 960 can be a C2 flat washer NAS 620, stainless steel McM-C No. 90945A105 2362; compression spring 970 can be 120 OD, 0.016 wire diameter stainless steel McM-C No. 9435K13 2633; fork 930 can be a die cast or molded fork and can either be made from material such as stainless steel or autoclavable engineering plastic or other material suitable for hygienic dental purposes; bar 940 can be ⅛″ square and can have a 2-56 tapped hole minimum 3/16″ deep.

The socket connection mechanism shown in the illustrated embodiment of FIG. 36 depicts a rotation mechanism connected directly to the forked members. In some embodiments, however, the bar can be split into a first operative piece connected to the collimator end and a second operative piece extending from mounting pin arrangements. The first and second operative pieces can be joined by a rotation mechanism similar to that shown in FIG. 36. In some embodiments the rotation mechanism can be placed relatively close to the collimator end, and in other embodiments the rotation mechanism can be placed closer to the image receptor, as disclosed in the illustrated embodiment. In still other embodiments, the rotation mechanism can be located at the collimator end of the x-ray device wherein the bar is connected to the rotation mechanism. In this way the entire bar is rotatable.

The embodiments of mounting pin arrangements and rotatable mounting pin arrangements described above can be made from a material that can be sterilized. The arrangements may also be made of radiotransparent or radiopaque materials.

In some applications, mounting pin arrangements may not provide the most comfort for patients when using image receptors mounted on x-ray devices. When image receptors mounted to the mounting pin arrangements are inserted into a patient's mouth, the patient typically will bite down to secure the image receptor in place while an x-ray image is being taken. When a patient bites down on the image receptor end, the patient's teeth may, in some situations, come into contact with the bar or other structure associated with the mounting pin arrangements. These situations may lead to discomfort for some patients. In some applications, it may be advantageous to provide some type of covering, padding, or cushioning mechanism such that a patient's bite on or near the bar or mounted pin arrangement mechanism may be as comfortable as possible.

In one embodiment of a cushioning mechanism, FIG. 38 shows bite block 990 prior to installation on forked member 870 that is in use with an x-ray device having a quadrilateral shaped collimator 991. The arrangement of forked member 870 provides an area to insert a soft bite block for patient comfort. In some applications, bite block 990 may also assist in keeping the image receptor end stable when in use in a patient's mouth. Bite block 990 is adapted to be received on forked member 870 and in some applications can be securely fashioned to forked member 870 to prevent swallowing. Slots 992 are formed into surface of bite block 990 and are adapted to receive at least a portion of forked member 870. When bite block 990 is fixed into place on forked member 870, bite block 990 can provide a cushioning surface when a patient bites down. Bite block 990 can be made from any material suitable to provide comfort to a patient including, but not limited to, cotton, foam, rubber, and any type of soft, durable resin. Bite block 990 can also be made of a material that can be sterilized in preparation for use on other patients. Furthermore, bite block 990 can be made in different thicknesses and softnesses for different procedures.

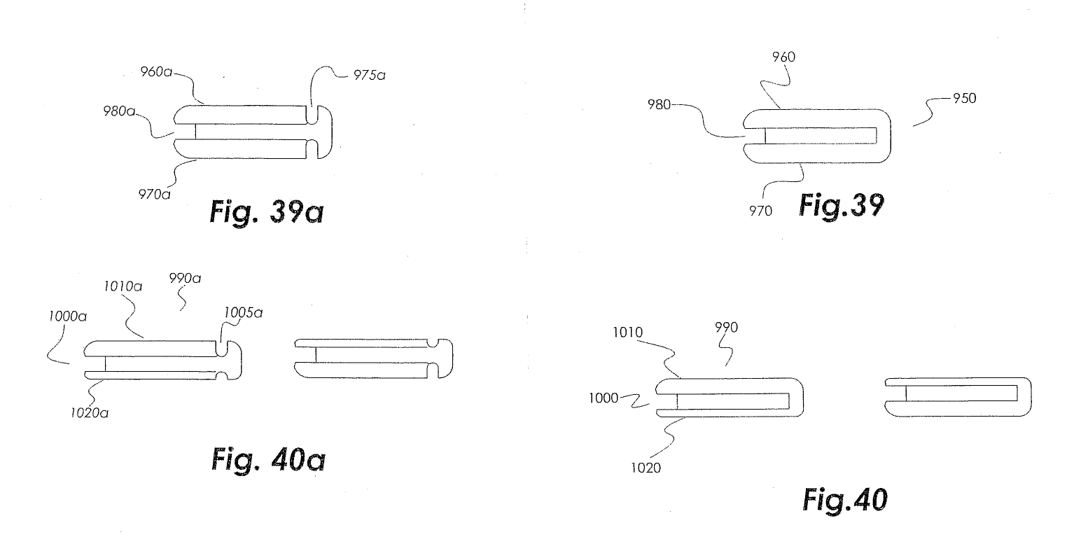

FIG. 39 shows one embodiment of bite block indicated generally at 950. Bite block 950 can have a symmetrical top side 960 and bottom side 970. Aperture 980 is adapted to receive a forked member, such as forked member 870 in FIG. 38.

In an alternative embodiment, FIG. 39a shows a bite block indicated generally at 950a. Bite block 950a can have symmetrical top and bottom sides, 960a and 970a respectively, but can also have slot 975a shaped to receive the edge of an image receptor. Slot 975a is useful in maintaining image receptor alignment in the receptor end as well as assisting in locking the bite block into the image receptor. Aperture 980a is adapted to receive a forked member, such as forked member 870 in FIG. 38.

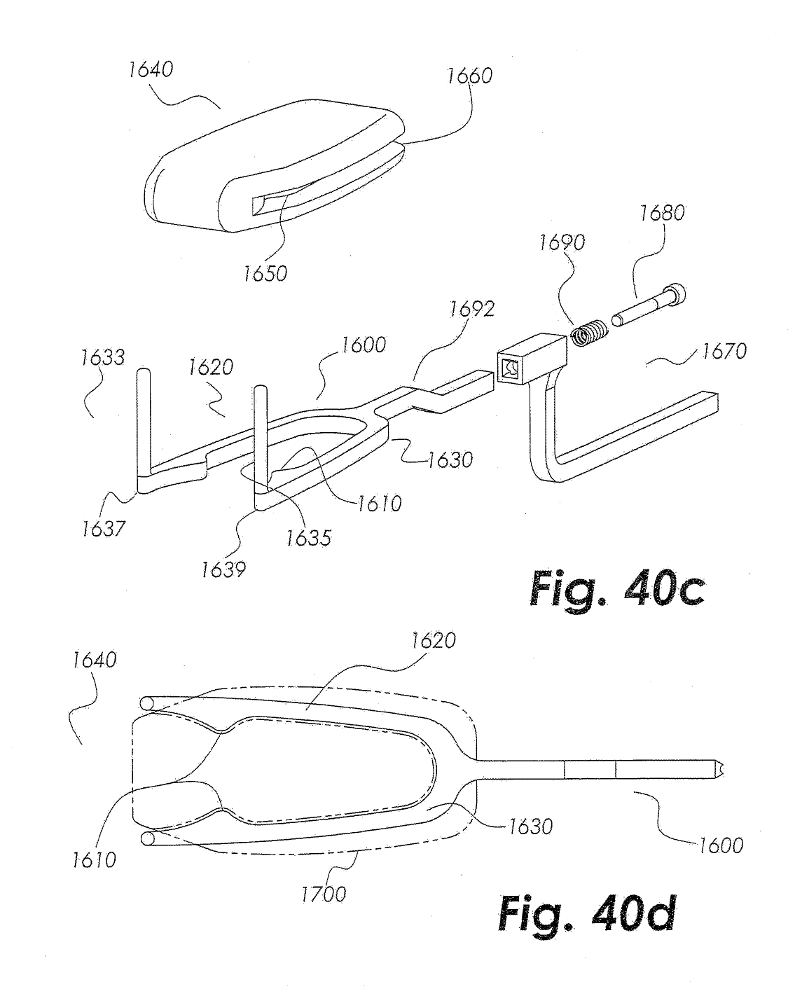

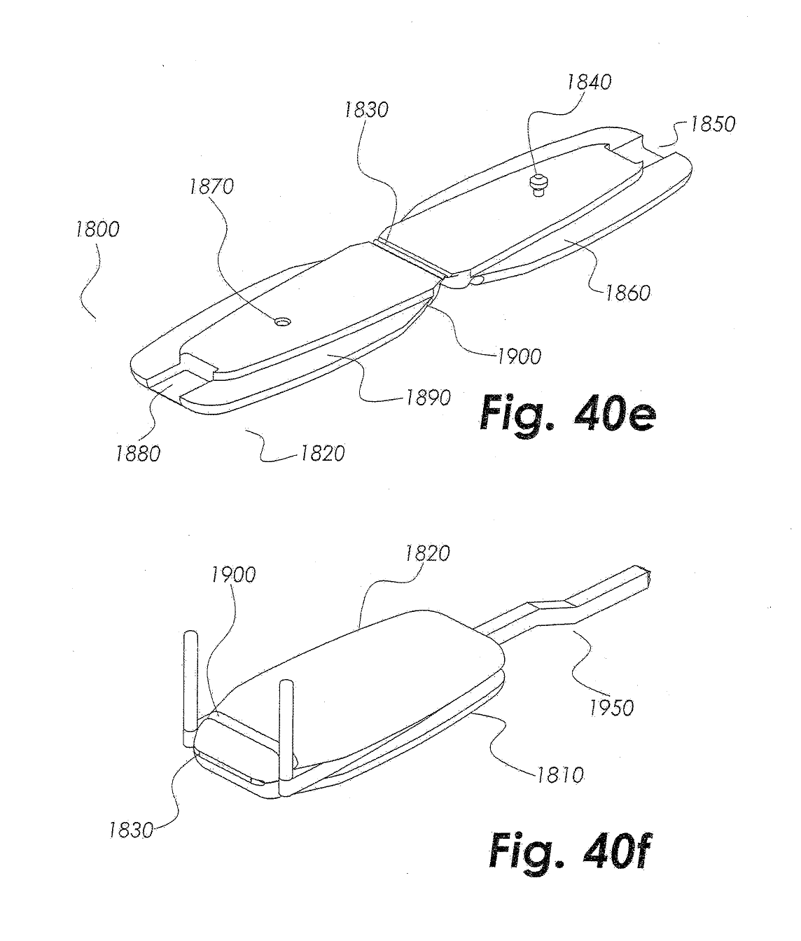

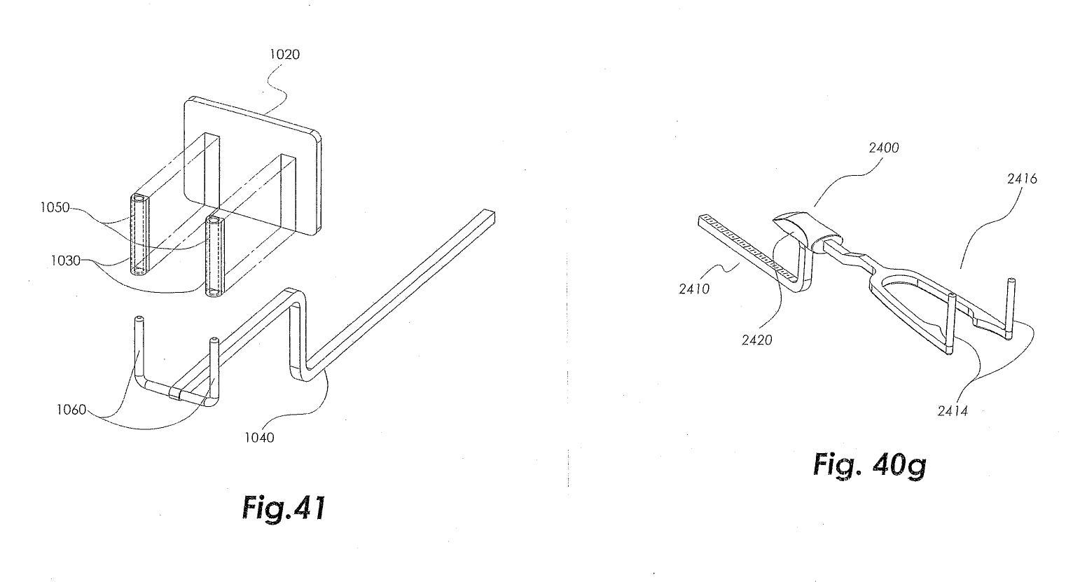

FIG. 40 shows another embodiment of a bite block indicated at 990. Bite block 990 is shown in a non-symmetrical configuration having an aperture 1000 that is asymmetrical between a top surface 1010 and a bottom surface 1020. Other geometries of bite blocks are also possible. In other embodiments, bite blocks can be made to adapt to any variety of mounting pin arrangements.

In an alternative embodiment, FIG. 40a shows a bite block indicated at 990a. Bite block 990a is shown in a non-symmetrical configuration having a top surface 1010a larger than a bottom surface 1020a. Aperture 1000a is adapted to receive a forked member such as forked member 870 in FIG. 38. Slot 1005a is shaped to receive the edge of an image receptor. Slot 1005a is useful in maintaining image receptor alignment in the receptor end as well as assisting in locking the bite block into the image receptor.

In some situations cotton rolls are used in certain situations to move a film holder into certain positions. Cotton rolls may not lead to precise placement of an image receptor because the cotton roll can move in relation to the image receptor at anytime after the item is placed by the dentist or other medical personnel. Bite blocks of various sizes, including various thicknesses, provided as described above can eliminate the need to use cotton rolls as the blocks can keep component parts in sync. Furthermore, bite blocks as provided above may not move in the patient's mouth during use. The bite blocks may also be discarded after use or may alternatively be sterilized and/or recycled for further use.

Other types of bite block arrangements are also possible given configuration changes in the forked member. Some embodiments of the systems described above provide for friction fit between the forked members and bite blocks. In some applications it may be advantageous to provide for a mechanical fit between the bite block and forked member.

FIG. 40c depicts an embodiment of forked member 1600 having ears 1610 and elongated forks 1620 and 1630. Forks 1620 and 1630 can be elongated as shown in the illustrated embodiment to provide for greater flexibility in treating patients of various sizes. Forked member 1600 can be molded or cast. Forked members 1620 and 1630 are shown in the illustrated embodiment as mirror images of one another. Other embodiments of forked members 1620 and 1630, however, may include forked members that are not mirror images.

Posts 1633 and 1635 are shown as integral parts of forked member 1600 and include rounded lower surfaces 1637 and 1639 useful in minimizing patient discomfort. Rounded surfaces 1637 and 1639 include a geometry that provides for a smooth transition from the quadrilateral shape of forks 1620 and 1630 to the cylindrical shape of posts 1633 and 1635.

Ears 1610 are shown provided on elongated forks 1620 and 1630. In other embodiments ears 1610 may be provided on only one fork. Ears 1610 may take the form of a smooth protuberance as shown in the illustrated embodiment or may take the form of a prong or fish hook in other embodiments.

Ears 1610 are adapted to be received in complementary bite block 1640 thus providing a structural connection. Bite block 1640 includes offsets 1650 to receive ears 1610. Similar to other embodiments described above, slot 1660 is adapted to be received by forks 1620 and 1630.

Also depicted in FIG. 40c is socket mechanism 1670 comprised of a screw 1680 and spring 1690. In the illustrated embodiment, socket mechanism 1670 is adapted to permit rotation of forked member 1600 in four orientations.

Forked member 1600 also includes offset 1692 useful for providing clearance of forked member 1600 in various orientations. In other embodiments, offset 1692 may extend in the other direction such that posts 1633 and 1635 are lowered in relation to screw 1680. In still other embodiments, offset 1692 may be a greater or shorter distance than shown in the illustrated embodiment thus providing greater range of movement of when forked member 1600 is rotated to a new position. Offsets 1692 can be coupled with alignment rings having apertures specifically arranged to provide for horizontal and vertical images to be taken without the need to change equipment. Such alignment rings are described in greater detail hereinbelow.

FIG. 40d depicts bite block 1640 and forked member 1600 from FIG. 40c installed together. The edge 1700 of bite block 1640 is larger than either fork 1620 or 1630 so that a patient's teeth do not come into contact with either fork. Ears 1610 can be seen in FIG. 40d.

Turning now to FIG. 40e, bite block 1800 is shown having a first half 1810 and a complementary second half 1820. Both halves are connected by hinge 1830. Hinge 830 permits first half 1810 and second half 1820 to be selectively placed into proximity with one another. The surface of both halves can have matching cored grid area to reduce material cost. First half 1810 includes a mushroom button 1840, finger tip recess 1850 and lip 1860. Second half 1820 includes recess 1870 adapted to receive mushroom button 1840, finger tip recess 1880, lip 1890, and slot 1900.

When first half 1810 and second half are urged into contact, mushroom button 1840 is securely received in recess 1870 so as to discourage separation of the two halves. Finger recess 1850 and 1880 can be grabbed by an instrument or a person's fingers to overcome the mushroom button and force the first half 1810 and second half 1840 to separate. Furthermore, lip 1860 and 1890 create a recess configured to receive a forked member.

FIG. 40f depicts bite block 1800 mounted on forked member 1950. First half 1810 is shown on the bottom of second half 1820. When first half 1810 and second half 1820 are joined, lip 1860 and lip 1890 form a channel through which the forks of forked member 1950 can pass. Hinge 1830 can be seen joining the first half 1810 and second half 1820. Slot 1900 is adapted to receive an image receptor. Slot 1900 can be configured to receive and retain an image receptor using a friction fit. As with other embodiments of bite blocks disclosed hereinabove, bite block 1800 can be made of a material that can be sterilized and recycled for use in other patients.

In some applications it can be difficult to properly place the image receptor inside a patient's mouth. Physical manipulation is often required of the image receptor and or the bar of the x-ray device to accurately position the receptor. In some instances doctors or other medical personnel can manipulate the placement of image receptors by grasping and maneuvering the bar. The bar of some x-ray devices, however, can be made relatively small in keeping with reducing the device mass in and around patients' mouths. Therefore it can be difficult or cumbersome to grasp the bar. Structure can be used, however, to aid doctors or other medical personnel when grasping and maneuvering the bar.

FIG. 40g shows an embodiment of finger grip 2400 installed on bar 2410 of an x-ray device. As can be seen, image receptors can be configured to be slidingly received over posts 2414 of forked member 2416. Finger grip 2400 includes finger recesses 2420 useful for grasping by the fingers of doctors or other medical personnel and assist placement of image receptors in patients' mouths. Other devices can also be used to assist in grasping the bar or image receptor end of the bar. For example, finger grip 2400 can be designed to receive a medical instrument useful in maneuvering the bar, such as would be the case with a servo-actuated arm useful in remote imaging applications.

Returning briefly to FIG. 33 for reference, image receptors may be manufactured without having recesses 840. Such receptors can be referred to as recess-free receptors. In situations where recess-free receptors are available it may be desirable to provide systems and mechanisms that adapt these receptors to the mounting pin arrangements such as those described above. Systems and mechanisms for adapting recess-free receptors to the mounting pin arrangement can be sold, for example, as add-on kits to accompany the sale of the receptors from the manufacturer, or can be sold as retrofit kits.

In one embodiment, FIG. 41 shows image receptor 1020 prior to installation of molded blocks 1030. As can be seen, image receptor 1020 lacks internal recesses necessary for direct mounting to the holder end of bar 1040. To remedy the absence of internal recesses, molded blocks 1030 are formed with passageways 1050 which are configured to receive pins 1060 located at the holder end of bar 1040. Such molded blocks are useful for adapting recess-free image receptors to mounting pin arrangements. In the illustrated embodiment, passageways 1050 extend the entire length of molded block 1030. In other embodiments, passageways 1050 may only extend as far as necessary to receive mounting pin arrangements. Molded blocks 1030 can be attached to image receptor 1020 with pressure sensitive adhesive. Many other methods can also be used to attach molded blocks 1030 to image receptor 1020. By affixing molded blocks 1030 to the image receptor, consistent placement of the image receptor relative to the x-ray field can be provided thus mitigating the need for an alignment grid discussed above. Molded blocks 1040 can be made of any suitable radiotransparent or radiopaque material. Additionally and/or alternatively, molded blocks 1040 can be made of a material that permits molded blocks to be sterilized.

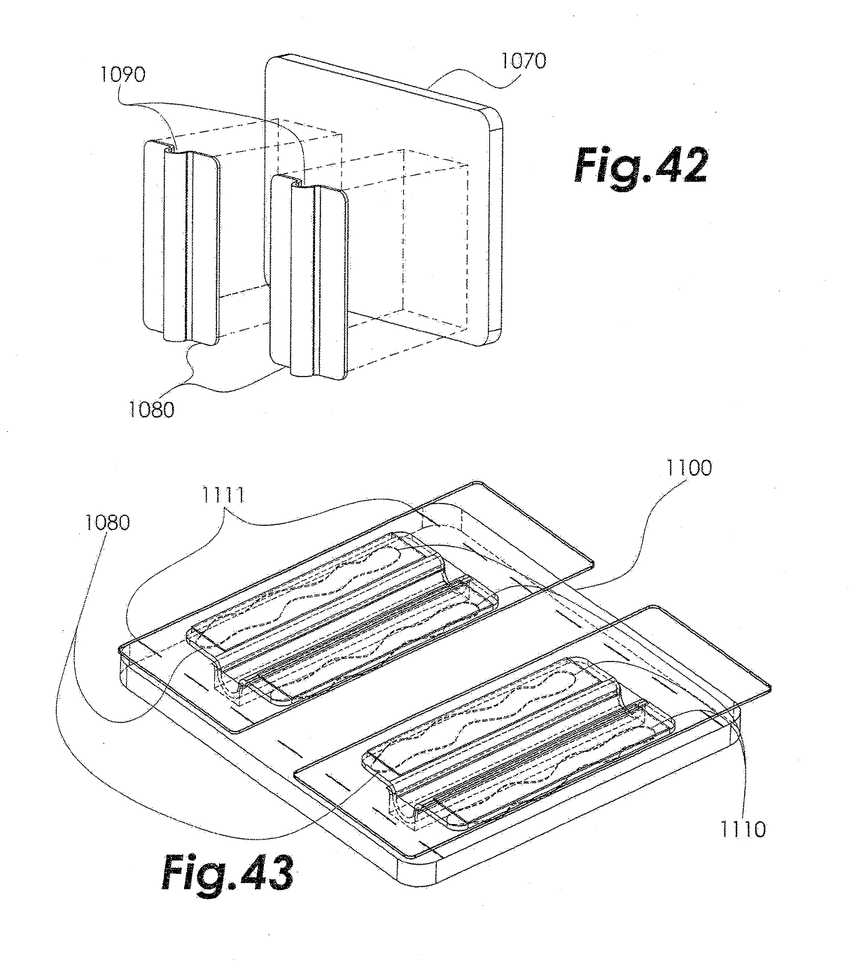

FIG. 42 shows another embodiment of a recess-free image receptor. Image receptor 1070 is shown prior to receiving vinyl pockets 1080. Pockets 1080 are elongate members having channels 1090 formed along their respective lengths. Channels 1090 are configured for use with mounting pin arrangements discussed above. It will be understood that channels 1090 need not extend the entire length of pockets 1080, but rather need only extend as far as necessary to receive mounting pins. Pockets 1080 can be attached to image receptor 1070 with adhesive and can be configured in any orientation appropriate to receive mounting pins. It will be understood that other methods can be used to attach vinyl pockets 1080 to image receptor 1070. As with the molded bocks 1030 of FIG. 41, by affixing vinyl pockets in the manner described, consistent placement of the image receptor can be provided thus mitigating the need for an alignment grid discussed above. Vinyl pockets 1080 can be made of any suitable radiotransparent or radiopaque material. Additionally and/or alternatively, vinyl pockets 1080 can be made of a material that permits the vinyl pockets to be sterilized.

In the applications described above, it is sometimes necessary to adhere individual blocks or vinyl pockets to the image receptor one at a time. Placement of the block or pocket on the image receptor one at a time may lead to misalignment between the opposing block or pocket on the other side of the image receptor possibly leading to difficulties in mounting the adapted image receptor to the mounting pins. In some situations, therefore, it may be advantageous to simultaneously apply a pair of molded blocks or vinyl pockets. It would be desirable, therefore, to provided systems and mechanisms for simultaneous application.

In one embodiment, FIG. 43 shows pockets 1080 pre-positioned on clear carrier card 1100 using alignment lines 1110. Alignment lines 1110 are provided to assist proper placement on carrier card 1100 so that pockets 1080 are in an orientation required to adapt an image receptor to the mounting pin arrangements described above. In other embodiments, pockets 1080 can be replaced with molded blocks or any other type of adapter useful in adapting recess-free image receptors to mounting pin arrangements. Pockets 1080 are tentatively retained, such as through a weak adhesive or any other suitable retention mechanism or force, to carrier card 1100 thereby forming an assembly. Once the assembly is oriented and pressed into place on the image receptor, adhesive 1110 attaches pockets 1080 to the image receptor. Other types of mechanisms or forces are contemplated to attach pockets 1080 to the image receptor. Using carrier card 1100 in this way encourages simultaneous application of the pockets 1080 to a surface of an image receptor thus encouraging a pair of pockets that are properly aligned relative to one another. After placement on the image receptor, carrier card 1100 can be removed by virtue of the fact that the tentative retention of the pockets by the carrier card is weaker than the adhesion of the pockets to the image receptor. Carrier card 1100 can then be disposed of or recycled for later use. In some embodiments, carrier card 1100 can be made of a material that can be sterilized.

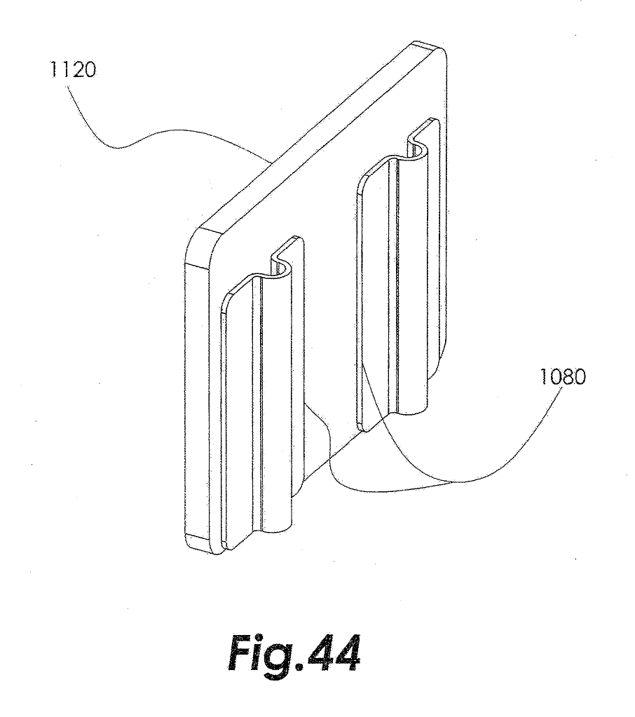

FIG. 44 depicts an image receptor 1120 having vinyl pockets 1080 adhered after use of a carrier card. As discussed above, molded blocks can be used in place of the vinyl pockets. It will be understood that various types of carrier card configurations are possible to pre-position and orient the systems and mechanisms necessary to adapt image receptors to the mounting pin arrangements described above.

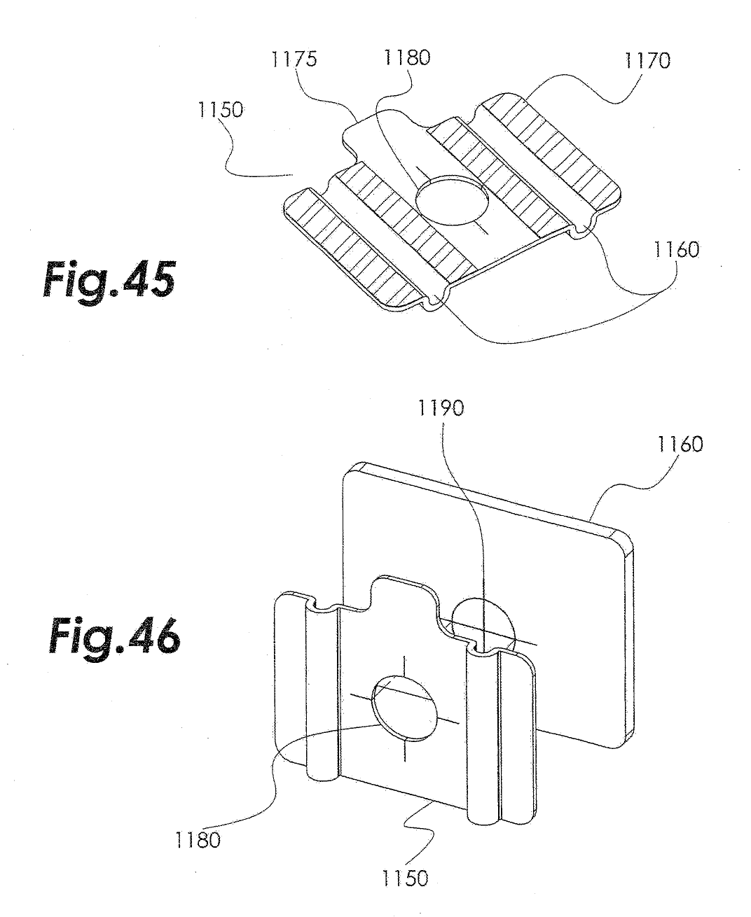

FIG. 45 shows another embodiment of systems and mechanisms for adapting recess-free receptors to the mounting pin arrangements previously discussed. Pocket assembly 1150 is shown in FIG. 45 having pockets 1160 formed to receive mounted pin arrangements. Surface 1170 can be affixed with an adhesive or other suitable attachment means to attach pocket assembly 1150 to a recess-free image receptor. Adhesives can be selected from a variety of substances, some of which include, but are not limited to, epoxy and glue. Pocket assembly 1150 has finger tab 1175 useful in handling pocket assembly 1150 without interference with adhesive. Pocket assemblies can be created without finger tab 1175, or can be created with other arrangements to provide for handling of the pocket assembly without interference with the adhesive or other suitable attachment mechanism. In some embodiments, target 1180 can be used to assist a person in properly orienting and affixing pocket assembly 1150 to a recessed-free image receptor. Alternatively and/or additionally, pocket assembly 1150 can be aligned to the bottom back edge of an image receptor. Pocket assembly 1150 can be made of any suitable radiotransparent or radiopaque material. Additionally and/or alternatively, pocket assembly 1150 can be made of a material that permits the pocket assembly to be sterilized.

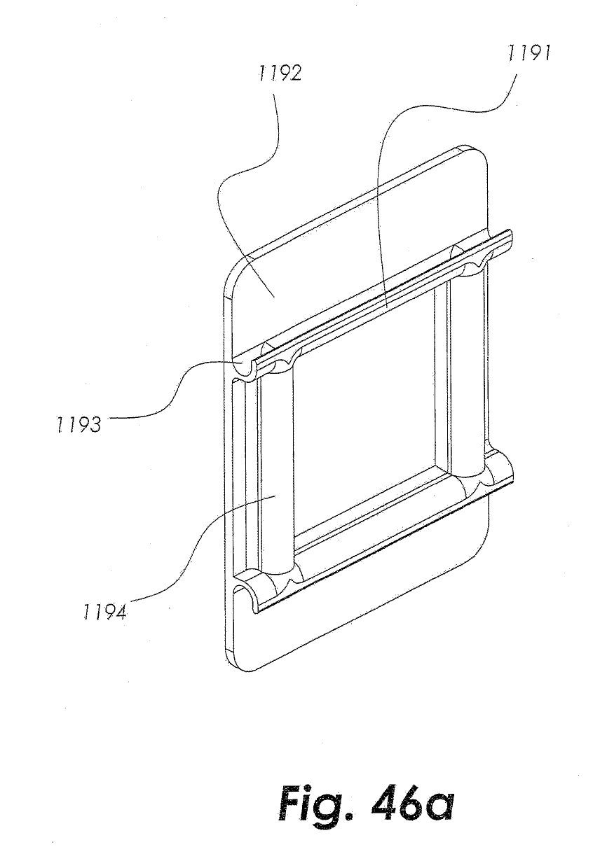

FIG. 46 shows assembly 1150 just prior to installation on image receptor 1160. As discussed previously, target 1180 can be aligned with receptor target 1190 on image receptor 1160. In some embodiments, pocket assembly 1150 can be made of any suitable substance and can be visually clear or radiopaque. In a clear embodiment, target 1180 and receptor target 1190 can be visually aligned such that pocket assembly 1180 is properly placed on image receptor 1160. In another embodiment, target 1180 can be a hole punched through pocket assembly 1150 such that a person aligning pocket assembly 1150 to receptor 1160 can use the hole to align the two. Pocket assembly 1150 can be attached in a variety of orientations allowing a non-square image receptor to be utilized with either the long edge or the short edge nearest the bar. Such orientations can be referred to as horizontal and vertical images.

FIG. 46a shows another embodiment of systems and mechanisms for adapting recess-free receptors to the mounting pin arrangements previously discussed. Lattice assembly 1191 is shown attached to image receptor 1192. Lattice assembly 1191 comprises a base and lattice channels 1193 and 1194 which are attached to the base. Lattice channels 1193 and 1194 are formed to receive mounting pin arrangements and are useful in providing a single adapter that permits placement of image receptor in a horizontal or vertical orientation prior to being slidingly received on mounting pin arrangements. For example, if a vertical image is desired then mounting pin arrangements can be inserted into channels 1194; likewise if a horizontal image is desired then mounting pin arrangements can be inserted into channels 1193. Lattice assembly 1191 can be made of any suitable radiotransparent or radiopaque material. Additionally and/or alternatively, lattice assembly 1191 can be made of a material that permits the lattice assembly to be sterilized.

In the applications described above, it is sometimes necessary to transport and store image receptor adapters. Some receptor adapters have pre-applied adhesive which may attract dirt or other particles if not properly shielded. Additionally and/or alternatively, the receptor adapters can be typically pre-sterilized and therefore are ready to be used in a doctor's office. Coverings can be provided to shield the receptor adapters thus protecting the adhesive and safeguarding the sterility of the adapter so that the adapters can be readily used.

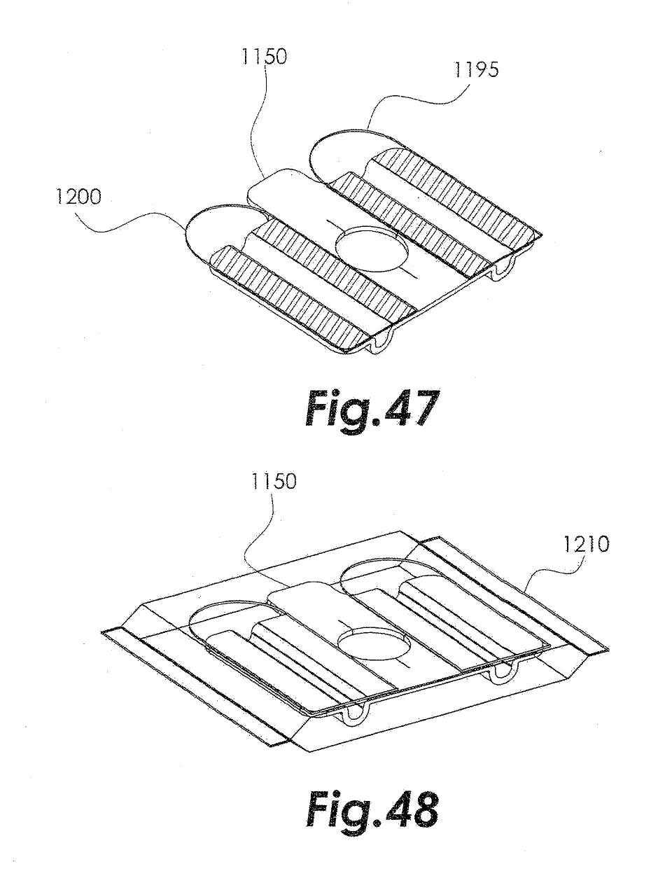

Turning now to FIG. 47, pocket assembly 1150 is shown having a cover 1195 attached to adhesive strips 1170. Covers 1195 can be used to transport and protect pocket assembly 1150 by discouraging adhesion to anything prior to removal of covers 1195. Covers 1195 can be removed just prior to installation of pocket assembly 1150 onto image receptor 1160 as shown previously in FIG. 46. Furthermore, covers 1195 can be made of any suitable material to promote temporary adhesion to adhesive strips 1170 yet allow covers 1195 to be easily removed from the adhesive strip when desired. Covers 1195 can have a thumb grab 1200 to allow easy removal from pocket assembly 1150. In some embodiments, covers 1195 can be sterilized and recycled for later use.

FIG. 48 shows an embodiment of pocket assembly 1150 packaged in a sterile pack 1210. Covers 1195 are shown adhered to pocket assembly 1150. In such a situation, sterile pack 1210, pocket assembly 1150, and covers 1195 can be sold in packs to dental offices, dental distributors, etc. Sterile pack 1210 allows pocket assembly 1150 to remain sterile and provides a package that can be handled freely and sold in bulk with relative ease. Alternatively and/or additionally, covers 1195 could be formed so as to envelop pocket assembly 1150 much like sterile pack 1210. In some embodiments, sterile pack 1210 can be made of a disposable material much like that of a wrapper for bandages. In other embodiments, sterile pack 1210 can take the form of a re-closable plastic bag such as that used for sandwiches. In still other embodiments, sterile pack 1210 can be made from a material that can be sterilized and recycled for later use.

Patient comfort and safety can be further enhanced through systems and mechanisms that confine the bar and holder end of an x-ray device to as small a volume as possible. Some types of image receptors (e.g., digital CCDs) require electrical connections between the receptor and the x-ray devices. The electrical connection in some applications can take the form of a thin electrical cable or wire. These types of electrical connections increase the amount of materials present in a patient's mouth during a procedure. If the wire or cable were not secured at intermediate positions of the bar and instead were allowed to dangle loosely, the effective volume of the x-ray device may be increased which may lead to added discomfort and reduced patient safety. It would be beneficial, therefore, to provide systems and mechanisms for retaining the cable or wire as closely as possible to the bar so as to minimize the effective volume of the x-ray device.

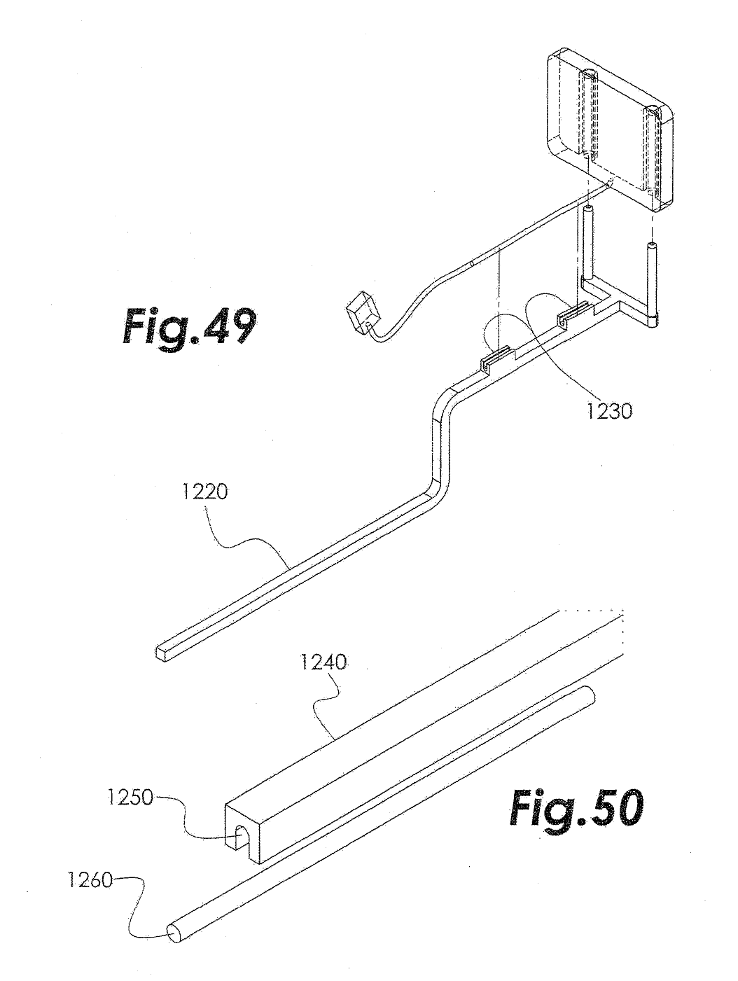

Turning now to FIG. 49, bar 1220 is shown having wire holder clips 1230. Clips 1230 can be circular in shape having an inner diameter that permits a snug-fit of the image receptor cable or wire, and a circumference that extends over at least half the wire or cable so as to retain the cable or wire in place. In this way the wire or cable of, for example, a digital CCD image receptor can be retained at certain points along the length of the bar so as to minimize the effective volume of the x-ray device. Clips 1230 allow for the cable or wire to be quickly replaced, moved, or repaired without the need for excessive disassembly of the x-ray unit. In some embodiments, clips 1230 can be removable from bar 1220. Removable clips permit the adaptation of a bar having no clips to be able to receive and retain such wires. Alternatively and/or additionally, clips 1230 can be made of a material that can be sterilized.

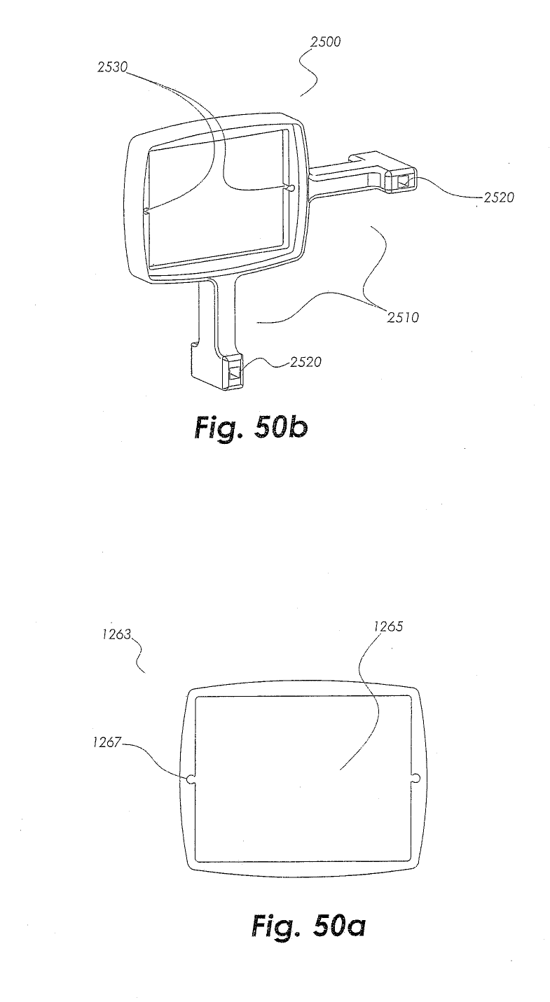

In yet another embodiment, FIG. 50 shows a shaped alignment bar 1240. The alignment bar 1240 provides a U-shaped channel 1250 adapted to receive cable 1260. Channel 1250 can extend along substantially the entire length of bar 1240 from the adapter end to the holder end. In this way cable 1260 of, for example, a digital CCD image receptor can be substantially retained along the length of the bar so as to minimize the effective volume of the x-ray device. Channels provided as described above allow the cable or wire to be easily replaced if needed without the need to excessively disassemble the x-ray unit. It will be appreciated that other systems and mechanisms can be used to at least partially retain a wire or cable of an image receptor. In some embodiments, bars having channels 1250 can be made of a material that can be sterilized.

In some applications, the thin electrical wire or cable leading from certain types of image receptors may be retained by the bar but may also be further routed and retained interior to the collimator tube. Lead wires or cables can be routed interior to the collimator tube along an inward facing surface. Lead wires or cables routed internal to the collimator tube may provide for an aesthetically pleasing and relatively clean exterior surface. Operators of x-ray devices may find that having wires routed internal to the collimator tube reduce interference with operation of the unit.

FIG. 50a shows the front view of a collimator tube 1263 having a generally rectangular shape. Aperture 1265 extends along the length of collimator tube 1263 and acts to collimate the x-radiation. In the illustrated embodiment, divot tracks 1267 are shown formed in to the inside surface of collimator tube 1263. As used herein, ‘divot tracks’ comprise techniques useful in retaining lead wires within a collimator tube such as, but not limited to, channels formed along either the entire inside surface of the collimator tube, or alternatively channels formed along a shorter length of the collimator tube. Divot tracks can also include channels supplemented with and or replaced by retaining clips. In some embodiments divot tracks may not include a channel and instead rely solely on retaining clips. In those applications where a channel is used, divot tracks include those techniques wherein a lead wire can be press fit within a track. Other techniques that retain lead wires within a collimator tube are contemplated. Divot tracks 1267 can be created by molding or machining, among other techniques. In some applications, divot tracks need appear on only one side of collimator tube. In other applications, multiple divot tracks may be desired. In some embodiments, divot tracks may be located on the exterior surface of the collimator tube.

In some applications, collimator tubes may have been adapted to receive an alignment ring. In some applications it would be beneficial to provide a cutout in the alignment ring similar to divot tracks discussed above. It will be understood, however, that the alignment ring can be configured to accommodate a lead wire without the need to also provide divot tracks internal to the collimator tube.

FIG. 50b shows alignment ring 2500 having arms 2510, apertures 2520, and divots 2530. Similar to divot tracks in collimator tubes disclosed above, divots 2530 are useful to retain the lead wires for certain types of image receptors. As used herein, ‘divots’ comprise techniques useful in retaining lead wires routed near alignment rings such as, but not limited to, channels formed in the inside or outside surface of the alignment ring. In some applications divots can be supplemented and/or replaced with retaining clips. In some embodiments divots may not include a channel and instead rely solely on retaining clips. Where a channel is used, divots include those techniques wherein a lead wire can be press fit within a track. Other techniques that retain lead wires within a collimator tube are contemplated. Divots 2530 can be created by molding or machining, among other techniques. In some applications, divots need appear on only one side of the alignment ring. In other applications, multiple divots may be desired. In still other applications, divot tracks may be located on the outside of the alignment ring such that the lead wire is routed external to the collimator.

In applications using image receptors having no need for wires or cables (e.g., x-ray film) it may be unnecessary to provide systems and mechanisms such as those shown in FIG. 49, FIG. 50, FIG. 50a or FIG. 50b. Instead certain bars can be provided for x-ray film and other bars provided for digital CCD image receptors. It may be advantageous, however, to provide systems and mechanisms of common bar arrangements that are suitable for different applications. Such would allow an operator of the x-ray device to procure one bar that has multiple uses. To provide a bar that is useful in multiple applications, bar arrangements can be provided that eliminate the need for external wires or cables and instead provide for the transmission of electrical signals from the image receptor to electrical devices through the existing structure of the bar and through the pin-type arrangements or film alignment bar arrangements.

Returning for the moment to FIG. 33, one or both of pins 820, or a portion of pins 820, can be made from a suitably conductive material. Likewise, one or both of recesses 840 can provide for an appropriate electrical connection between image receptor 830 and one or both of pins 820. Furthermore, bar 800 can provide for an electrically conductive path from one or both of pins 820 to associated electrical devices such as a wireless transmitter. Viewing the system as a whole, an electrical signal generated by image receptor 830 can be communicated through one or more of recesses 840 to one or more of pins 820 and along bar 800 to an associated electrical device thereby eliminating the need for a wire or cable. Any or all of the elements involved in the transmission of electrical signals can be at least partially insulated. It will be understood that various types of electrical connections are possible to route electrical signals from the image receptor, through the pins and along the bar to associated electrical devices.

In certain applications it can be advantageous to provide systems and mechanisms for use with x-ray devices that provide enhanced recordation of x-ray images captured by image receptors. In particular, systems and mechanisms can be used to provide multiple records of x-ray images resulting from a single use of an x-ray device. In some situations, insurance companies may require physical copies of x-ray images while doctors and dentists may find it convenient for diagnostic purposes to manipulate digital versions of x-ray images. Certain embodiments of systems that provide multiple records of x-ray images offer additional benefits of less exposure time to x-radiation for the patient and less time involved for the dental staff to produce multiple x-ray images. It can be possible to provide images of different types by using a combination of different types of image receptors. For example, x-ray film can be used at the same time as a digital CCD image receptor by placing the film in front of the CCD image receptor. It will be understood that in this situation the lead backing, if any, of the x-ray film must be removed prior to use to permit transmission of x-radiation through film to the digital CCD.

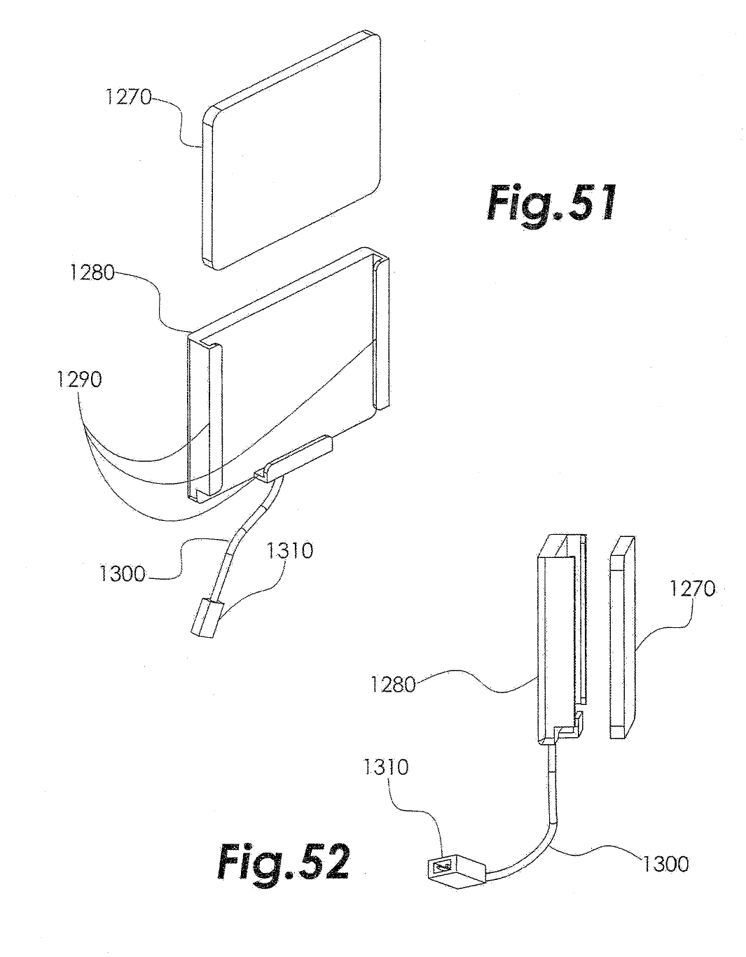

FIG. 51 shows x-ray film 1270 prior to placement in a digital sensor 1280. Sensor 1280 has flanges 1290 that are shaped to receive and retain x-ray film 1270 for use. It will be understood that techniques other than flanges can be used to align and retain x-ray film 1270 relative to sensor 1280. It will also be understood given the discussion above that x-ray film 1270 may not have a lead backing to permit digital sensor 1280 to capture an image. It can be possible, however, to operate the x-ray device with a lead backed x-ray film and digital CCD with the understanding that digital sensor 1280 may not provide a suitable image. Cable 1300 is attached to sensor 1280 and has connector 1310 capable of forming electrical connection with other devices or connectors. It will be further understood that the systems and mechanisms disclosed hereinabove useful for mounting image receptors, such as the mounting pin arrangements, can be used in the combination of x-ray film 1270 and digital sensor 1280.

FIG. 52 is a side view of a completed assembly of x-ray film 1270 and digital sensor 1280.

In some applications it is advantageous to provide systems and mechanisms for use with x-ray devices that provide improved protection from excessive or unneeded x-radiation. The National Council on Radiation Protection (NCRP) issues criteria directed to radiation exposure of patients. Commercially available collimator tubes sometimes come in fixed lengths and fixed aperture geometries that, in some uses, may fail to provide adequate protection consistent with NCRP criteria. For example, collimator tube geometries may create a beam of x-radiation within which image receptors must be located with particularity (e.g., distance from the end of the tube) so as to avoid unnecessary exposure to x-radiation. Failure to locate the image receptor with particularity may lead to increased x-radiation exposure. As will be appreciated, particular placement of image receptors can sometimes prevent flexibility of image receptor placement often beneficial in treating patients. Collimator tube geometries can be created, however, to provide greater flexibility in placement of image receptors while maintaining improved protection from excessive or unneeded x-radiation.

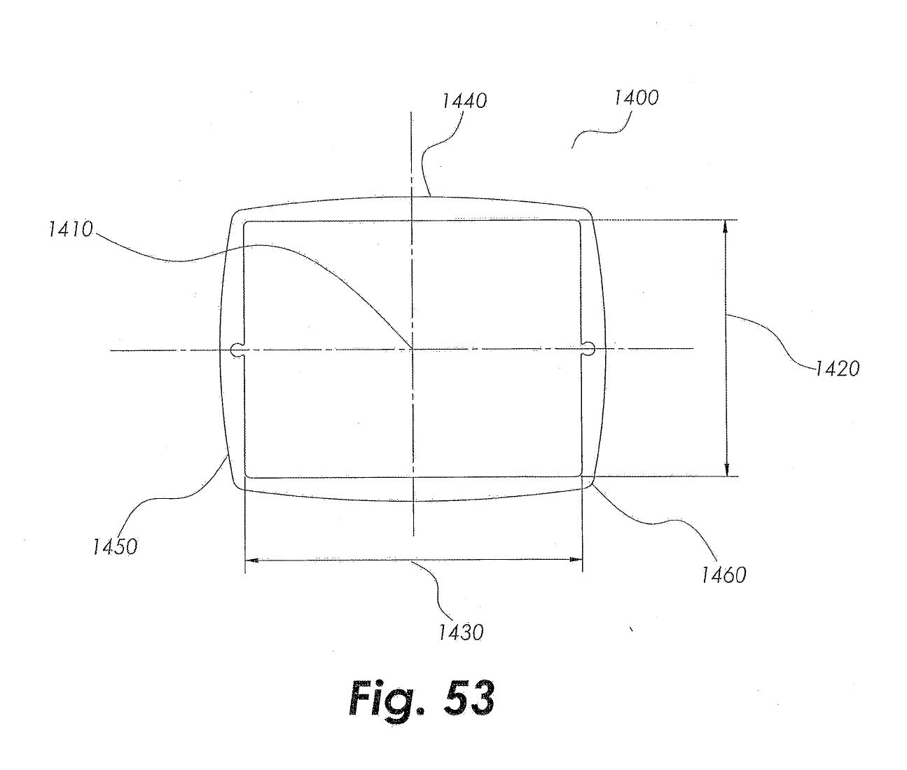

FIG. 53 shows one embodiment of a quadrilateral shaped collimator tube indicated generally at 1400. Collimator tube 1400 is a generally symmetrical tube geometry having center 1410. Collimator tube 1400 has an inside distance in the first direction 1420 of 1.328″ and an inside distance in the second direction 1430 of 1.734″. Additionally, collimator tube 1400 has outside surfaces on opposing sides in the first direction with a radius of curvature 1440 of 6.045″ and outside surfaces on opposing sides in the second direction with a radius of curvature 1450 of 3.542″. Collimator tube 1400 also has outside corners having a radius of curvature 1460 of 0.062″.

Collimator geometries of the illustrated embodiment in FIG. 53 produce x-ray fields much smaller than commercially available collimator tubes. Table 1 compares the actual x-ray fields of the embodiment illustrated in FIG. 53 against the actual x-ray fields of existing rectangular collimator tubes. A collimator length of 12″ was used to calculate the values contained in Table 1. The comparisons are made at various image receptor distances from the x-ray source; the numbers in parentheses are distances as measured from the end of the collimator. In addition to the aforementioned comparisons, the 2% Source-to-Image Receptor-Distance (SID) are also included in the table. The 2% SID values are based on an image receptor having a size of 1.210″×1.610″. The 2% SID requirements are included to highlight the greater suitability and flexibility of the embodiment shown in FIG. 53 to accommodate different distances of image receptor to x-ray source.

As can be seen, a linear relationship exists between the x-ray field sizes and the distance of the image receptor to the x-ray source. The linear relationship owes to the relative positioning of the x-ray source and the exit aperture of the collimator tube. Other configurations of x-ray source, collimator exit aperture and collimator length can yield similar results. For example, the length and exit aperture geometry of a collimator can be changed to narrow or broaden the values found in Table 1. In particular, a longer collimator will result in a narrower beam. A narrower exit aperture also results in a narrower beam. Because the values in Table 1 have a linear relationship, linear interpolation of the values demonstrate that the embodiment of FIG. 53 is capable of meeting the 2% SID requirements at a distance of about 5.8″ from collimator tube to image receptor. The existing collimator tubes, in contrast, fail to meet the 2% SID requirements even at a 2″ distance.

TABLE 1

| Distance of image | | | |
|---|---|---|---|
| receptor to x-ray | | Actual x-ray field | Actual x-ray field |
| source; (distance | 2% SID x-ray | of the embodiment | of existing rectangular |
| from end of collimator) | field requirement | in FIG. 53 | collimator tubes |
| 14″ (2″) | 1.490″ × 1.890″ | 1.199″ × 1.549″ | 1.545″ × 2.047″ |
| 16″ (4″) | 1.530″ × 1.930″ | 1.370″ × 1.770″ | 1.366″ × 2.340″ |
| 18″ (6″) | 1.570″ × 1.970″ | 1.542″ × 1.992″ | 1.987″ × 2.630″ |
| 20″ (8″) | 1.610″ × 2.010″ | 1.713″ × 2.213″ | 2.208″ × 2.925″ |

As will be appreciated, image receptors of the illustrated embodiment in FIG. 53 placed 2″ from the collimator may underutilize the image receptor but still provide coverage within the 2% SID requirement. Image receptors placed at 5.8″ also meet the 2% SID requirement as previously discussed and provide for greater utilization of the image receptor. Greater flexibility in placement of image receptors is thus attained with the embodiment of FIG. 53.

Image receptors having dimensions different than 1.210″×1.610″ can also be used. It will be appreciated that the geometry of the collimator can be changed to accommodate different image receptor dimensions while at the same time providing for useful coverage within at least 5.8″ of the collimator tube.

In other applications it is advantageous to provide systems and mechanisms for use with x-ray devices that provide improved protection from excessive or unneeded x-radiation. In particular, the composition of the collimator tube can be selected to impede and/or reduce the transmission of x-radiation through the material that composes the collimator tube.

In one such embodiment of a composition useful in reducing the transmission of x-radiation, collimator tubes can be composed of a tungsten filled polymer which has similar properties to lead in controlling emissions of x-radiation. One embodiment of a tungsten filled polymer is compound 705ZD70 which is within the acrylonitrile-butadiene-styrene chemical family and available from ECOMASS of 4101 Parkstone Heights Dr. Suite 380, Austin, Tex. 78746-7482. The formula for the compound is proprietary but some properties are known: the compound has a high specific gravity, is a tungsten filled ABS compound, is rigid, and possesses good toughness. Collimator tubes made from such a compound can be extruded or injection molded. As listed in the data sheet from ECOMASS, the compound has an ISO 1183 specific gravity of 3.00 g/cc; ISO tensile strength of 30 MPa; ISO 178 flexural modulus of 2,900 MPa; ISO 180/A Izod impact strength of 6.0 kJ/m2; melt temperature range of 480-500 F.; mold temperature range of 100-150 F.; mold shrinkage rage (4 mm) of 0.004-0.006 mm/mm; and pre-drying conditions of 2-4 hours at 160-180 F., desiccant bed dryer recommended. The composition described herein may also be used for other structures such as the x-ray head housing. Other compositions may also be used to reduce the transmission of x-radiation through the collimator tube.

X-ray images can be useful in a variety of medical field. For example, x-ray images are useful in dental offices for both health checkups and the diagnosis and treatment of dental diseases. Such uses of dental x-rays can often require images to be taken from a number of different perspectives that include anterior, posterior, bitewing, and endodontic. Such perspectives also include upper and lower teeth. X-ray images taken from each of these perspectives sometimes requires unique configurations of alignment rings and image receptors. Accordingly, equipment can be designed for each.

When switching between perspectives, changes to equipment or changes in relative positioning of the equipment can sometimes be required. Such changes require medical personnel to devote time and energy to accomplishing the change. For example, medical personnel may be required to replace the alignment ring, re-orient the new alignment ring to a new position, and/or change alignment arms. Systems and mechanisms can be created, however, to reduce the variety of equipment needed and/or reduce the need to change positioning of the equipment when switching from one perspective to the other. Such reductions in effort improve the delivery of medical care by reducing the time needed in some applications for medical personnel to mechanically operate equipment and instead provide more time for substantive medical care.

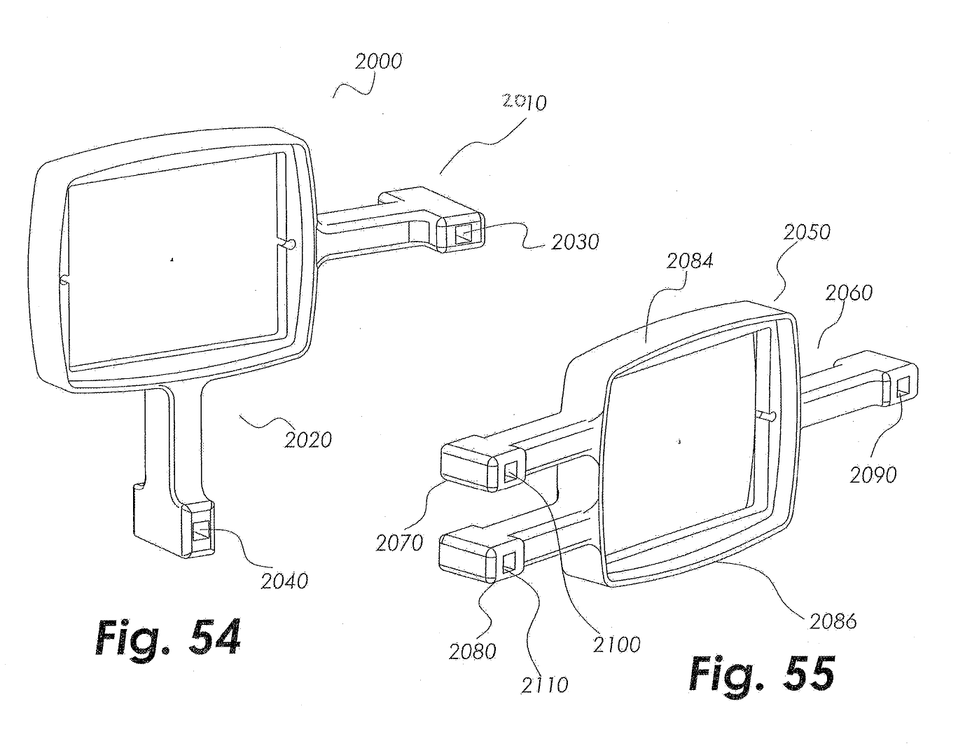

Turning now to FIG. 54, alignment ring 2000 is shown having a vertical alignment arm 2010 and a horizontal alignment arm 2020 useful for coupling a bar of a x-ray device (discussed in an embodiment above) to the collimator of the device (also discussed in an embodiment above). Vertical alignment arm 2010 has aperture 2020 adapted to slidingly receive the bar. Similarly, horizontal alignment arm 2020 has aperture 2040 adapted to slidingly receive the bar. A benefit of using the alignment arms of FIG. 54 is that a single bar of an x-ray device may be used for both horizontal and vertical images. Vertical alignment arm 2010 and horizontal alignment arm 2020 can be configured in other embodiments to allow connection of the bar of an x-ray device in a similar manner to a socket as opposed to apertures 2030 and 2040 in the illustrated embodiment. Other connections are also contemplated, such as magnetic.

The embodiment disclosed in FIG. 54 shows arms disposed on adjacent sides of alignment ring 2000. In some applications it can be advantageous to provide alignment rings having arms arranged on opposite sides of the ring. In those applications it is either necessary to provide for two different bar arrangements to accommodate horizontal and vertical images, or alternatively, provide a bar having a selectively rotatable image receptor end such that the end can be rotated to accommodate a horizontal image when using the horizontal alignment arm, and a vertical image when using the vertical alignment arm.

FIG. 55 depicts an embodiment of an alignment ring 2050 having horizontal and vertical arms disposed on opposite sides of the alignment ring. Vertical alignment arm 2060 is shown disposed on one side of alignment ring 2050; horizontal alignment arms 2070 and 2080 are shown disposed on the other side. Alignment ring 2050 has two horizontal alignment bars to permit image receptors to be configured such that the base of the image receptor is in proximity to either the first side 2084 or the second side 2086 of alignment ring 2050. Much as in the embodiment depicted in FIG. 54, vertical alignment arm has aperture 2090 and horizontal alignment arms 2070 and 2080 have apertures 2100 and 2110 respectively.

Apertures 2090, 2100, and 2110 are adapted to slidingly receive the bar of an x-ray device. As will be appreciated, use of the bar having a selectively rotatable image receptor end (discussed in an embodiment above) facilitates use of vertical and horizontal alignment arms. For example, when the bar is slidingly removed from the vertical alignment arm and re-inserted into the horizontal alignment arm, rotation of the image receptor end places the image receptor back into the field of x-rays emanating from the collimator tube aperture. Alternatively, separate bars can be used to capture horizontal and vertical images using the horizontal and vertical alignment arms. Apertures 2090, 2100, and 2110 can be replaced in other embodiments to allow connection of the bar of an x-ray device manner similar to a socket. Other connections are also contemplated, such as magnetic.

In some applications it may be advantageous to provide for sliding adjustment of alignment arms on a given alignment ring so that the bar and image receptor can be slidingly adjusted in at least one dimension for any given procedure. Alignment arms that can be slidingly adjusted provide for additional flexibility to the doctor or other medical personnel to place the image receptor end in a desired location.

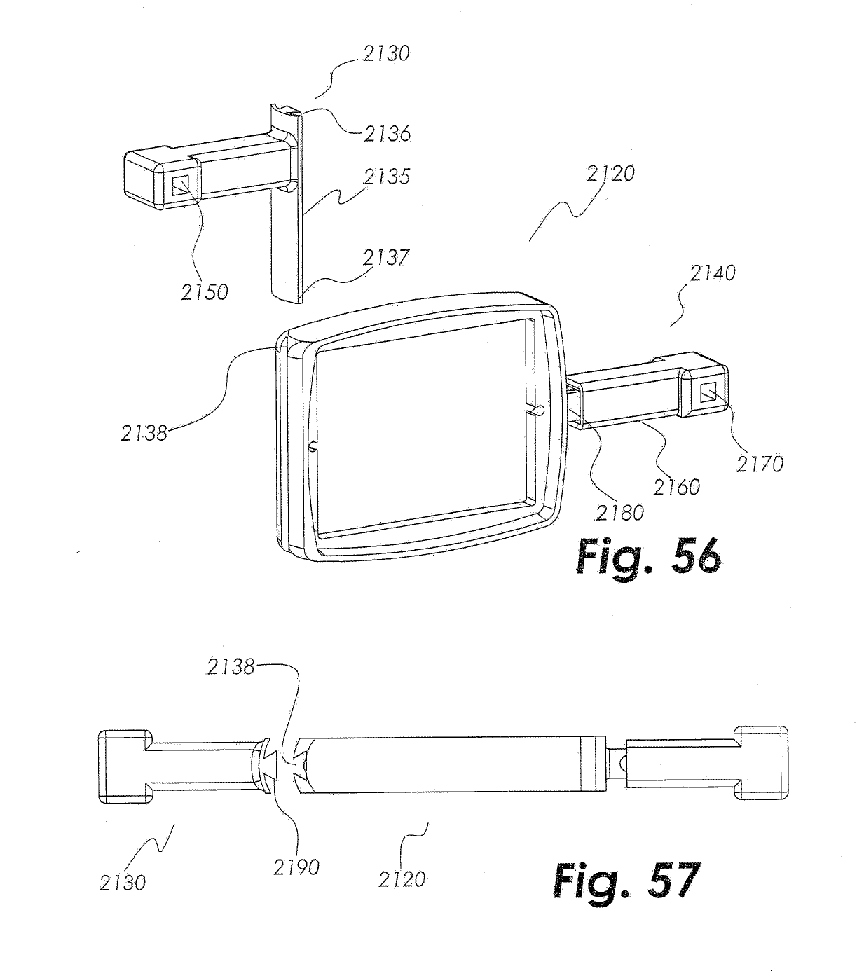

FIG. 56 depicts alignment ring 2120 having slidable horizontal alignment arm 2130 and slidable vertical alignment arm 2140. Slidable horizontal alignment arm 2130 is comprised of base 2135 and aperture 2150. Base 2135 has first end 2136 and second end 2137 and is adapted to be slidingly received in groove 2138 of alignment ring 2120. Groove 2138 is formed in alignment ring 2120. A sliding arrangement such as that just discussed can be referred to as a ‘base and groove sliding arrangement.’ Slidable horizontal alignment arm 2130 is arranged to allow for aperture 2150 to be slidingly adjusted in one dimension along the height of alignment ring 2120. A slidingly adjustable arm permits flexible placement of the bar of the x-ray device for any particular use.

In the illustrated embodiment, second end 2137 is received in groove 2138 and rests at the bottom of alignment ring 2120 such that aperture 2150 is oriented towards the top of alignment ring 2120. As used herein, the terms ‘top’ and ‘bottom’ are not meant to connote a spatial relationship but, rather, is meant to be understood as describing the upper and lower part, respectively, of the figures as displayed on the drawing sheets as the drawing sheets are normally oriented for reading and comprehension. In some applications, slidable horizontal alignment arm 2130 can be flipped around wherein first end 2136 is received into groove 2138 and rests at the bottom of alignment ring 2120 such that aperture 2150 is oriented towards the bottom of alignment ring 2120. As will be understood, aperture 2150 can be placed into any position along the height of alignment ring 2120 to allow for a variety of positions capable of receiving the bar of an x-ray device.

Slidable vertical alignment arm 2140 is comprised of sleeve 2160 and aperture 2170. Sleeve 2160 is adapted to be slidingly received over post 2180. Post 2180 is attached to alignment ring 2120. Sleeve 2160 is therefore arranged to allow for aperture 2170 to be slidingly adjusted in one dimension along the length of post 2180 which permits useful placement of the bar of the x-ray device to be adjusted for any particular use. Such an arrangement can be referred to as a ‘post and sleeve sliding arrangement.’ In the illustrated embodiment, sleeve 2160 is located in close proximity to alignment ring 2120. In some applications, sleeve 2160 may be oriented away from alignment ring 2120.

In some embodiment, the base and groove arrangement of the horizontal alignment arm in the illustrated embodiment can be used instead on the vertical alignment arm. Likewise in some embodiments, the post and sleeve arrangement of the vertical alignment arm in the illustrated embodiment can be used for the horizontal alignment arm. In still other embodiments, an arm of an alignment ring can be configured having the base and groove sliding arrangement of slidable horizontal alignment arm 2130 coupled with the post and sleeve sliding arrangement of slidable vertical alignment arm 2140. In this way, the aperture of such an arm can be adjusted in two dimensions, that is, in one dimension of the base and groove and the other dimension of the post and sleeve. Such two dimensional adjustments allow for enhanced flexibility.

Turning now to FIG. 57, the top view is shown of alignment ring 2120 discussed above in FIG. 56. Slidable horizontal alignment arm 2130 is shown having tongue 2190. Tongue 2190 is adapted to be received in groove 2138. In some embodiments, the tongue can be attached to the alignment ring while the groove is formed in the base.

Slidable alignment arms disclosed above are useful in adapting bars of x-ray devices to a variety of positions relative to alignment rings. Alignment arms can be adapted to provide further flexibility in placement of the bars by providing rotating or pivoting arms.

FIG. 58 discloses alignment ring 2200 having pivoting alignment arm 2205 comprising aperture 2210, pivot 2220, and base 2230. Aperture 2210 is shown oriented in a first position towards the top of alignment ring 2200. Base 2230 is pivotally connected to alignment ring 2200 by pivot 2220. Pivot 2220 is aligned along the centerline of base 2230 in the illustrated embodiment so that when pivoting alignment arm 2205 is rotated, aperture 2210 will be aligned in a second position oriented towards the bottom of alignment ring 2200. The second position is the mirror-opposite of the first position in the illustrated embodiment. In other embodiments, pivot 2220 may be located off-centerline of base 2230 such that the first and second positions are not mirror images of one another.

In some applications it would be desirable to provide enhanced flexibility in placement of alignment arms. Such would be possible by combining features disclosed hereinabove.

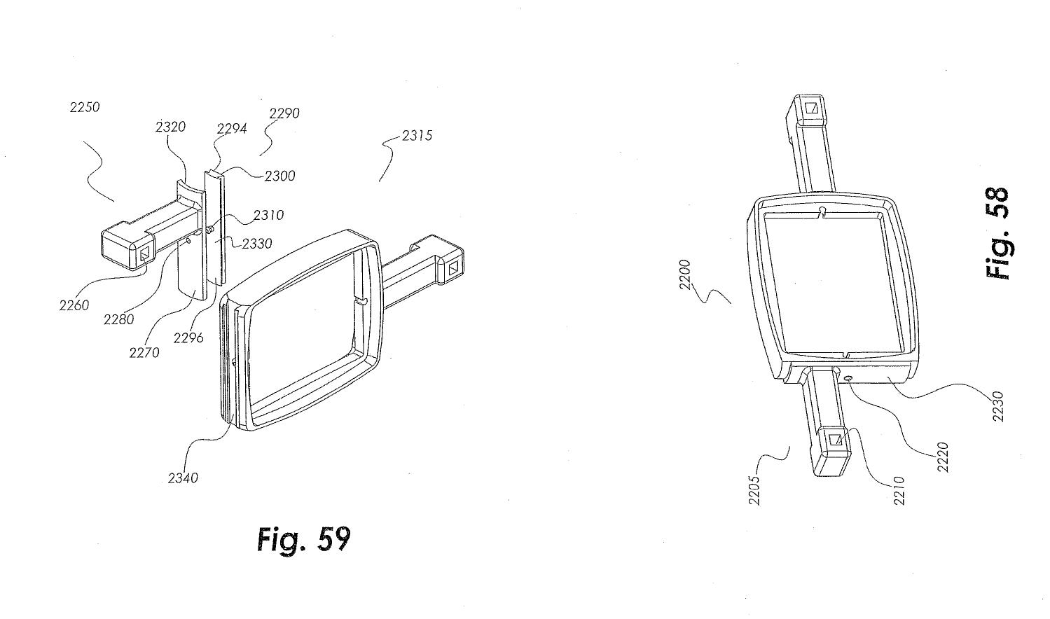

FIG. 59 displays alignment arm 2250 comprised of aperture 2260, base 2270, and base aperture 2280. Also shown in FIG. 59 is sliding adapter 2290 having a first end 2294 and second end 2296. Sliding adapter 2290 comprises tongue 2300 and pivot 2310. Base aperture 2280 and alignment arm 2250 are configured to pivotally connect to sliding adapter 2290 by receiving pivot 2310. Base aperture 2280 is aligned along the centerline of base 2270 in the illustrated embodiment so that when pivoting alignment arm 2250 is rotated, aperture 2260 will be aligned in a second position oriented towards the bottom of alignment ring 2315. The second position is the mirror-opposite of the first position in the illustrated embodiment because base aperture 2280 is aligned along the centerline. In other embodiments, base aperture 2280 may be located off-centerline of base 2270 such that the first and second positions are not mirror images of one another

The underside 2320 of alignment arm 2250 is adapted to cooperate with surface 2330 of sliding adapter 2290. Tongue 2300 is adapted to be received by groove 2340 of alignment ring 2350.

In the illustrated embodiment, second end 2294 is received in groove 2340 and rests at the bottom of alignment ring 2350 such that aperture 2260 is oriented towards the top of alignment ring 2350. As stated previously, the terms ‘top’ and ‘bottom’ are not meant to connote a spatial relationship but, rather, are meant to be understood as describing the upper and lower part, respectively, of the figures as displayed on the drawing sheets as the drawing sheets are normally oriented for reading and comprehension. In some applications, sliding adapter 2290 can be flipped around wherein first end 2294 is received into groove 2340 and rests at the bottom of alignment ring 2350 such that aperture 2260 is oriented towards the bottom of alignment ring 2350. As will be understood, aperture 2260 can be placed into any position along the height of alignment ring 2350 to allow for a variety of positions capable of receiving the bar of an x-ray device.

In some applications is may be desired to further reduce the number of devices needed to capture x-ray images. The variety of perspectives required to render medical services necessitate using a variety of devices. As such, bars having a variety of shapes and sizes may be needed to adequately address the variety of perspectives. Some bar arrangements having geometric flexibility, such as those disclosed herein having rotatable image receptor ends, offer the possibility of reducing the total number of bars required to accommodate the different perspectives. To realize even greater reduction in the number of devices, alignment rings can be designed to cooperate with certain types of bars having geometric flexibility.

FIG. 60 shows an alignment ring 2600 having aperture 2605, alignment arm 2610, arm aperture 2620, stub aperture 2630, and magnets 2640. Magnets 2640 are useful to attach and remove alignment ring 2600 from the end of a collimator tube. Arm aperture 2620 is adapted to receive a bar of an x-ray device. In some applications, arm aperture 2620 is adapted to receive a bar having a rotatable image receptor end. Alignment arm 2620 is designed such that when the bar is placed into arm aperture 2620, the image receptor is aligned within the field of x-radiation emanating from aperture 2605.

Stub aperture 2630 is adapted to receive a bar of an x-ray device similar to arm aperture 2620. In some applications, stub aperture 2630 is adapted to receive a bar having a rotatable image receptor end. Placement of stub aperture 2630 relative to the center of alignment ring 2600 is designed such that when the bar having a rotatable image receptor end is placed into stub aperture 2630, and once the image receptor end is rotated toward aperture 2605, the image receptor is then aligned within the field of x-radiation emanating from aperture 2605.

The geometry of stub aperture 2630 and arm aperture 2620 relative to the center of alignment ring 2600 can be designed so as to cooperate with a single bar having a rotatable image receptor end such as shown in FIG. 36, FIG. 40c, FIG. 40g, and FIG. 46a. For example, when the bar is inserted into arm aperture 2620, an image receptor can be placed on the image receptor end of the bar to align with aperture 2600 to capture a vertical image. If so desired, the same bar can be removed from arm aperture 2620 and reinserted into stub aperture 2630. As will be appreciated, simply reinserting the bar into stub aperture 2630 results in a mis-alignment of the image receptor. Therefore, to align the image receptor with aperture 2605 after relocating the bar, the rotatable image receptor end of the bar is rotated 90 degrees thus providing capture of a horizontal image. It can be seen, therefore, that a single bar having a rotatable image receptor end can be used to capture horizontal and vertical images.

In some embodiments of alignment rings, alignment arms such as that described in alignment arm 2610 can be located on all four sides of a quadrilateral shaped alignment ring. Likewise, stub apertures such as that described by stub aperture 2630 can be located in multiple places around the alignment ring. Alternatively and/or additionally, base and groove arrangements and post and sleeve arrangements can be provided for alignment arm 2610 and stub aperture 2630.

In applications where an x-ray image is taken having the top and bottom teeth in the same image, flat structures such as bite-wings are provided that connect to an image receptor and allow a patient to bite down thus securing the image receptor in place. The bite-wing is normally disposed at an angle such as 90 degrees from the image receptor. Bite-wings can be created that are integral and/or connected to mounting pin arrangements as disclosed hereinabove.

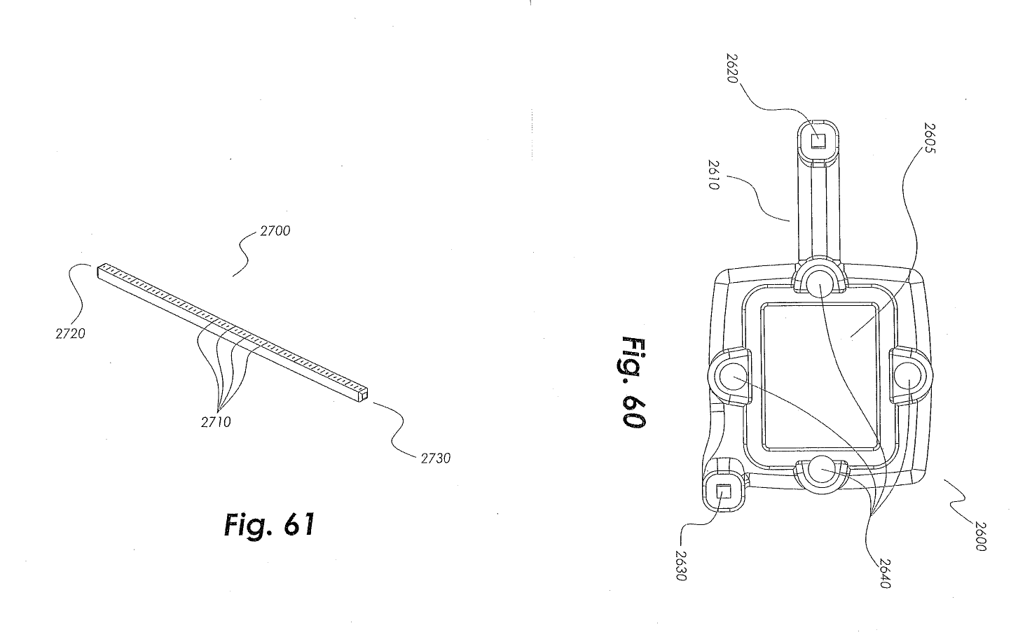

FIG. 61 discloses a bar 2700 adapted for use in x-ray devices and useful for coupling an image receptor holder to a collimator of an x-ray device. Bar 2700 is shown as generally straight and is useful to accommodate horizontal and bite-wing film/sensor holders. As discussed hereinabove, markings 2710 are useful in noting the relative location of an image receptor holder to the collimator tube. Such relative locations are useful to doctors and other medical personnel to capture successive images taken from the same general location over a period of time so as to track the progress of medical treatment. Bar 2700 has a first end 2720 and a second end 2730. In some embodiments, first end 2720 can be connected to the collimator tube or to an alignment ring of the collimator tube; the second end 2730 in these embodiments can be connector to an image receptor holder or similar platform. In other embodiments, first end 2720 can be connected to an image receptor holder or similar platform and the second end 2730 can be connected to the collimator tube or an alignment ring of a collimator tube.

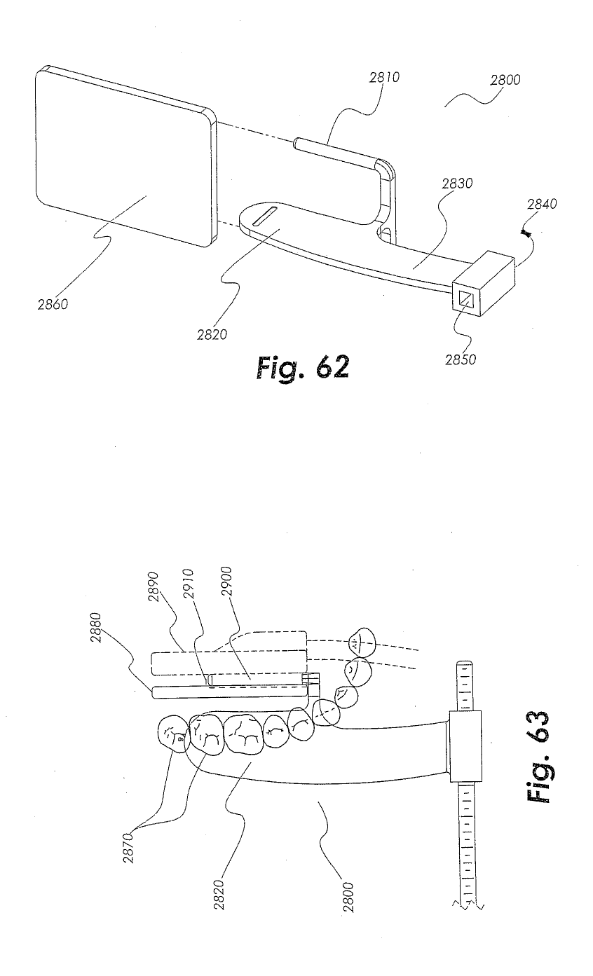

FIG. 62 discloses bitewing 2800 having mounting pins 2810, tongue 2820, support arm 2830, and adapter 2840. Mounting pins 2810 are useful for receiving image receptor 2860. Image receptor 2860 can be formed having apertures useful in receiving mounting pins 2810, or can be adapted to receive pins 2810 using any of the methods hereinabove disclosed. When received on mounting pins 2910, image receptor 2860 will be partially above and partially below tongue 2820 such that when a patient bites down on bitewing 2800, image receptor 2860 can be used to capture an image of a patient's upper and lower teeth. Adapter 2840 has aperture 2850 useful in receiving bars of an x-ray devices such as those disclosed hereinabove. Bitewing 2800 can be made of a material that can be sterilized so that bitewing 2800 can be recycled for later use. In some embodiments, bitewing 2800 can be made of a radiotransparent or radiopaque material.

FIG. 63 shows a top view of bitewing 2800 from inside a patient's mouth. The relative positioning of bitewing 2800 to patient's teeth 2870 can be seen. Tongue 2820 resides between a patient's upper and lower teeth and provides a surface on which a patient can bite down. Bitewing 2800 is thus securely received in a patient's mouth when teeth 2870 from a patient's upper and lower set of teeth bear down on tongue 2820. Film pack 2880 and digital sensor 2890 are shown attached simultaneously to mounting pins 2900. Adapter 2910 is configured much as in the embodiments disclosed above wherein image receptors lacking apertures are adapted to be received on mounting pins. In the illustrated embodiment, adapter 2910 has a surface for receiving film pack 2880, a surface for receiving digital sensor 2890, and recesses adapted to be received by mounting pins 2900. In this way a single adapter, such as adapter 2910, can be used to mount multiple image receptors on to a mounting pin arrangement.

Some bitewing arrangements are configured having an integral assembly of mounting pins and tongue. In some applications it may be desirable to provide detachable mounting pin arrangements. Such would be beneficial if, among other reasons, a mounting pin is broken or in needed of repair. Instead of disposing of the entire bitewing assembly, the mounting pin assembly itself could be replaced. The same holds true for damage to the bitewing. If a bitewing become broken or is in need of repair, the mounting pin arrangements could be salvaged and used on a new bitewing.

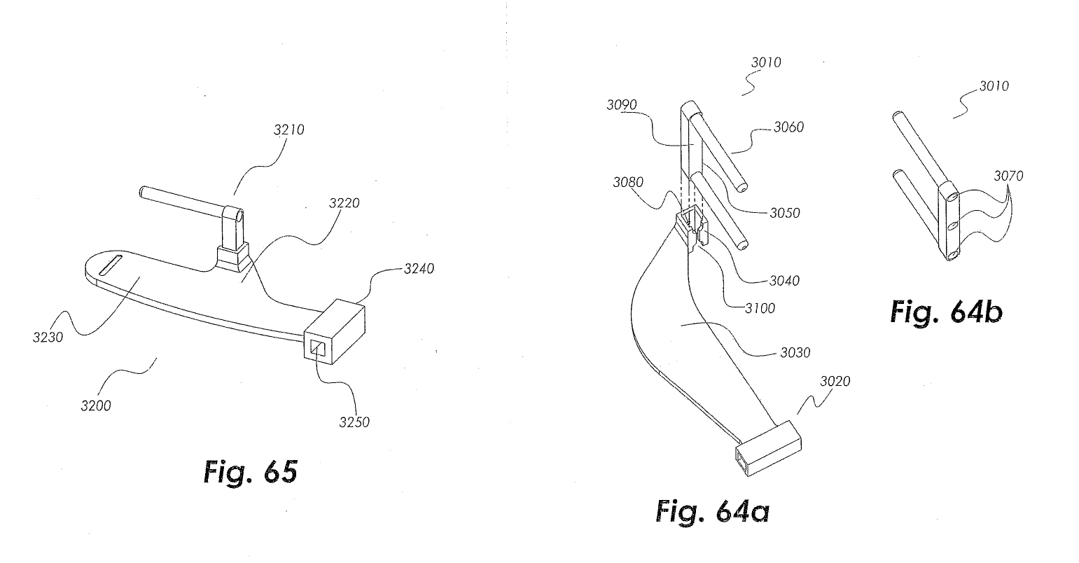

FIGS. 64a and 64b show an embodiment of bitewing 3000 and replaceable mounting pin 3010. Bitewing 3000 is adapted to receive replaceable mounting pin 3000 and comprises adapter 3020, tongue 3030, and connector 3040. Adapter 3020 has aperture sized to receive the bar of an x-ray device much as in FIGS. 62 and 63 above. Tongue 3030 is configured so as to provide a platform on which a patient can bite down and secure an image receptor. Connector 3040 is adapted to receive base 3050 of replaceable mounting pin 3010 such that replaceable mounting pin 3010 can be easily inserted and removed depending on the needs of a doctor or other medical personnel. Replaceable mounting pin 3010 has pins 3060 adapted to receive an image receptor.

Replaceable mounting pin 3010 has concave recesses 3070 adapted to receive convex structure 3080 on the inside surface of connector 3040. When base 3050 of replaceable mounting pin 3010 is inserted into connector 3040, convex structure 3080 is received into concave recesses 3070 thereby discouraging movement of replaceable mounting pin 3010 relative to bitewing 3000. In some embodiments, base 3050 can have convex structure attached to pin surface 3090 such that convex structure is adapted to be received in cutout 3100 formed in connector 3040. Other types of connection mechanisms between replaceable mounting pins and bitewings are contemplated such as, but not limited to, magnetic. Replaceable mounting pin 3010 can be made of a material that can be sterilized so that replaceable mounting pin 3010 can be recycled for later use. In addition, replaceable mounting pin 3010 can be made of a radiotransparent or radiopaque material.

Turning now to FIG. 65, bitewing 3200 is shown connected to replaceable mounting pin 3210. Bitewing 3200 includes raised holder 3220, tongue 3230 and adapter 3240. Adapter 3240 has aperture 3250 adapted to receive the bar of an x-ray device. Similar to bitewing embodiments disclosed above, tongue 3230 provides a platform on which a patient's teeth can bite in to so as to secure an image receptor in place during an x-ray procedure. Raised holder 3220 is configured to hold mounting pin 3210 in place and is provides a rounded, smoothed surface transition from tongue 3230. Rounded and smoothed surfaces can be used to ease patient comfort when taking x-ray images.

Embodiments of systems described herein may be combined to form an integrated system capable of achieving greater comfort and safety for patients of x-ray procedures. Reduced exposure to x-radiation is possible using novel collimator tubes, novel mounting pin arrangements, and novel inserts disclosed herein. Patients will appreciate greater comfort from reduced mass present in their mouth through novel mounting pin arrangements, novel forked member receptor holders, and novel bite blocks. Furthermore, accurate and repeatable x-ray images can be captured using the systems and mechanisms described herein giving medical personnel greater latitude in diagnostic and treatment options. It will be appreciated that systems and mechanisms described herein can be used on x-ray units as well as more permanent x-ray units typically used in dental and/or doctor offices. Any of the above embodiments may be combined with other embodiment to provide integrated x-ray systems providing doctors and other medical personnel greater flexibility in capturing x-rays as well as providing patients with greater comfort when taking x-ray images.

This disclosure serves to illustrate and describe the claimed invention to aid in the interpretation of the claims. However, this disclosure is not restrictive in character because not every embodiment covered by the claims is necessarily illustrated and described. All changes and modifications that come within the scope of the claims are desired to be protected, not just those embodiments explicitly described.

I claim:

1. An alignment device useful to interface a sensor with an X-ray device, the alignment device comprising:
a body constructed and arranged to be aligned with an emitting portion of the X-ray device;
a receptor holder constructed and arranged to removably couple the sensor; and
an alignment arm slidingly connected to the body, wherein the alignment arm is constructed and arranged to receive the receptor holder and wherein the alignment arm is constructed and arrange to be controllably movable with respect to the body to position the receptor holder in various different axial positions with respect to the body.

2. The alignment device of claim 1, wherein the receptor holder is controllably movable with respect to the alignment arm to position the sensor in various different longitudinal positions with respect to the body.

3. The alignment device of claim 1, wherein the body and the alignment arm together define a base and groove assembly.

4. The alignment device of claim 1, wherein the alignment arm further comprises a post and a sleeve assembly.
